SEQUENCE LISTING (16 sequences)

SEQ ID NO: 1 - protein, 4 aa, Artificial Sequence
  cyclic peptide
SWFR

SEQ ID NO: 2 - protein, 4 aa, Artificial Sequence
  cyclic peptide
SWFA

SEQ ID NO: 3 - protein, 4 aa, Artificial Sequence
  cyclic peptide
SWFK

SEQ ID NO: 4 - protein, 4 aa, Artificial Sequence
  cyclic peptide
SWFM

SEQ ID NO: 5 - protein, 4 aa, Artificial Sequence
  cyclic peptide
SWWR

SEQ ID NO: 6 - protein, 4 aa, Artificial Sequence
  cyclic peptide
SWYR

SEQ ID NO: 7 - protein, 4 aa, Artificial Sequence
  cyclic peptide
TWFR

SEQ ID NO: 8 - protein, 4 aa, Artificial Sequence
  cyclic peptide
SFWR

SEQ ID NO: 9 - DNA, 60 bp, Artificial Sequence
  synthetic primer
aagatcatatgacatcatcgtccacaacagcacctgcnnknnktgcatcagcggcgacag

SEQ ID NO: 10 - DNA, 63 bp, Artificial Sequence
  synthetic primer
aagatcatatgacatcatcgtccacaacagcacctgcnnknnknnktgcatcagcggcgacag

SEQ ID NO: 11 - DNA, 45 bp, Artificial Sequence
  synthetic primer
cttgccggtgctggccaggctgatcaggctgtcgccgctgatgca

SEQ ID NO: 12 - protein, 9 aa, Artificial Sequence
  cyclic peptide
VHNXXXCIS

SEQ ID NO: 13 - protein, 10 aa, Artificial Sequence
  cyclic peptide
VHNXXXXCIS

SEQ ID NO: 14 - protein, 9 aa, Artificial Sequence
  cyclic peptide
VHYXXXCIS

SEQ ID NO: 15 - protein, 9 aa, Artificial Sequence
  cyclic peptide
VHNXXXWIS

SEQ ID NO: 16 - protein, 9 aa, Artificial Sequence
  cyclic peptide
VHNXXXXCS

(19) United States
(12) Patent Application Publication
(10) Pub. No.: US 2006/0116316 A1
(43) Pub. Date: Jun. 1, 2006

(54) Heterocyclic inhibitors of IRES-mediated translation and methods of use thereof

(76) Inventors: Todd M. Kinsella, Redwood City, CA (US); Donald G. Payan, Hillsborough, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/251,268

(22) Filed: Oct. 14, 2005

Related U.S. Application Data
(60) Provisional application No. 60/619,420, filed on Oct. 14, 2004.

(51) Int. Cl. A61K 38/12; A61K 31/407; C07K 5/12

(52) U.S. Cl. 514/9; 530/317; 540/460

(57) ABSTRACT

The present invention provides heterocyclic compounds that exhibits IRES-inhibitory activity. The heterocyclic compounds generally a nine-membered ring of three repeating C—C—N subunits covalently bound through amide bonds, and variable side groups linked to a central carbon of each subunit. Formulations and kits containing the subject compounds are also provided.

### CROSS-REFERENCE TO RELATED APPLICATIONS

This application claims the priority benefit of U.S. provisional application No. 60/619,420, filed Oct. 14, 2004, which application is incorporated herein by reference in its entirety.

### FIELD OF THE INVENTION

The present invention relates generally to heterocyclic compounds, including those that have activity as anti-viral agents.

### BACKGROUND OF THE INVENTION

Hepatitis C virus (HCV) infection is an important clinical problem worldwide. In the United States alone, an estimated four million individuals are chronically infected with HCV. HCV, the major etiologic agent of non-A, non-B hepatitis, is transmitted primarily by transfusion of infected blood and blood products (Cuthbert et al., 1994, Clin. Microbiol. Rev. 7:505-532; Mansell et al., 1995, Semin. Liver Dis. 15:15-32). Prior to the introduction of anti-HCV screening in mid-1990, HCV accounted for 80-90% of posttransfusion hepatitis cases in the United States. Currently, injection drug use is probably the most common risk factor for HCV infection, with approximately 80% of this population seropositive for HCV. A high rate of HCV infection is also seen in individuals with bleeding disorders or chronic renal failure, groups that have frequent exposure to blood and blood products. In certain cases, HCV is sexually transmitted.

Acute infection with HCV results in persistent viral replication and progression to chronic hepatitis in approximately 90% of cases. For many patients, chronic HCV infection results in progressive liver damage and the development of cirrhosis. In patients with an aggressive infection, cirrhosis can develop in as little as two years, although a time span of 10-20 years is more typical. In 30-50% of chronic HCV patients, liver damage may progress to the development of hepatocellular carcinoma. In general, hepatocellular carcinoma is a late occurrence and may take greater than 30 years to develop (Bisceglie et al., 1995, Semin. Liver Dis. 15:64-69). The relative contribution of viral or host factors in determining disease progression is not clear.

Hepatitis C is an enveloped virus containing a positive-sense single-stranded RNA genome of approximately 9.5 kb. On the basis of its genome organization and virion properties, HCV has been classified as a separate genus in the family Flaviviridae, a family that also includes pestiviruses and flaviviruses (Alter, 1995, Semin. Liver Dis. 15:5-14). The viral genome consists of a lengthy 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation.

Translation of the HCV genome is initiated by a cap-independent mechanism known as internal ribosome entry. This mechanism involves the binding of ribosomes to an RNA sequence known as the internal ribosome entry site (IRES) (reviewed in Sonenberg & Meerovitch, 1990). As their names imply, these are sequences which enable ribosomes to bind to viral RNAs at internal sites rather than at the 5′-ends of these RNAs; having bound, the ribosomes can then migrate to the AUG initiator codon and begin translation. An RNA pseudoknot structure has recently been determined to be an essential structural element of the HCV IRES. As such, the IRES regulatory element is an essential component of viral translation and replication.

The mechanism by which HCV establishes viral persistence and causes a high rate of chronic liver disease has not been elucidated. Antiviral interventions to date have focused upon, for example, ribavirin and interferon-alpha (IFN-α)-based monotherapy and combination therapy. However, a significant portion of patients are not responsive to these therapies.

As such, a great need exists for new anti-HCV agents and new methods for combating HCV infections. The invention described herein meets this, and other needs.

### SUMMARY OF THE INVENTION

The present invention provides heterocyclic compounds that exhibits IRES-inhibitory activity. The heterocyclic compounds generally a nine-membered ring of three repeating C—C—N subunits covalently bound through amide bonds, and variable side groups linked to a central carbon of each subunit.

The IRES-inhibitory heterocyclic compounds may be generally described by the formula:

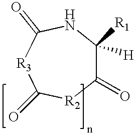

wherein n is 1 or 2,

R₁ is hydroxymethyl, 1-hydroxyethyl or thiomethyl;

R₂ and R₃ are, independently:

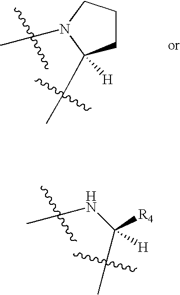

and R₄ is hydrogen, methyl, iso-propyl, iso-butyl, sec-butyl, methylthioethyl, benzyl, CH₂-linked 4-hydroxy-phenyl, CH₂-linked indole, hydroxymethyl, thiomethyl, ethanoic amide, propanoic amide, ethanoic acid, propanoic acid, 1-hydroxyethyl, 4-aminobutanyl, 4-(aminoiminomethyl)aminopropyl, hydroxymethyl, 1-hydroxyethyl, thiomethyl or CH₂-linked imidazole.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1A shows molecular structures of R groups that may be employed herein.

FIG. 1B shows molecular structures of 20 naturally occurring amino acids, which contain the R groups of FIG. 1A.

FIG. 2 schematically illustrates a method employed to assess cyclic peptide function.

FIG. 3 shows graphs of FACS data for exemplary peptides of the invention.

FIG. 4 shows graphs of mass spectrum data for c[TMW] isolated from cells (MS mode).

FIG. 5 shows a graph of mass spectrum data for c[TMW] isolated from cells (MS/MS mode).

FIG. 6 shows a graph of mass spectrum data for c[SPD] isolated from cells (LC/MS mode)

FIG. 7 shows a graph of mass spectrum data for c[SPD] isolated from cells (LC/MS/MS mode)

FIG. 8 schematically illustrates a method of isolating the subject peptides using n-butanol.

FIG. 9 shows graphs of mass spectrum data for c[TMW] isolated from cells by n-butanol extraction.

Before the present invention is described in more detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary and preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a candidate agent” includes a plurality of such candidate agents and reference to “the host cell” includes reference to one or more host cell and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

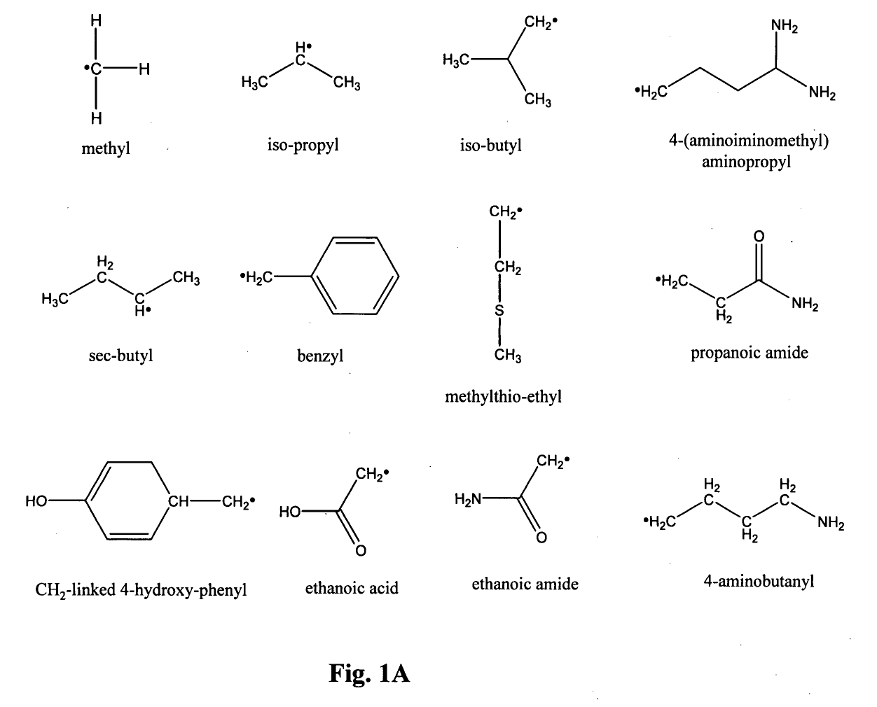

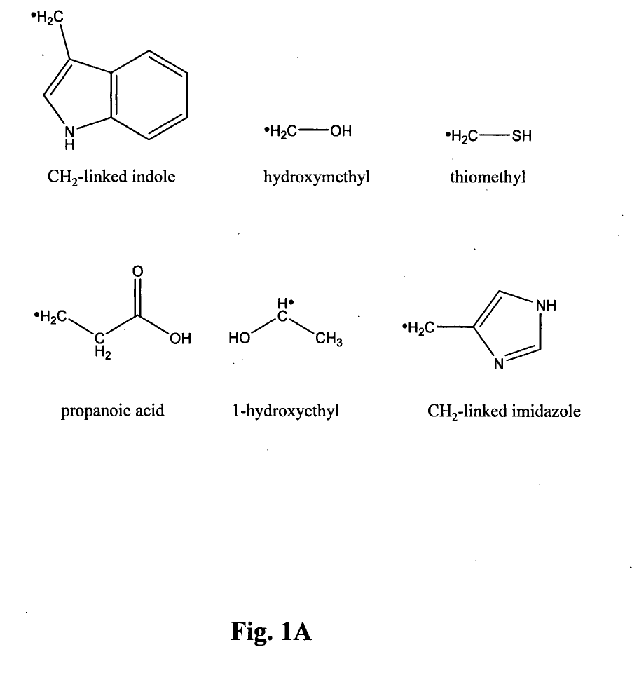

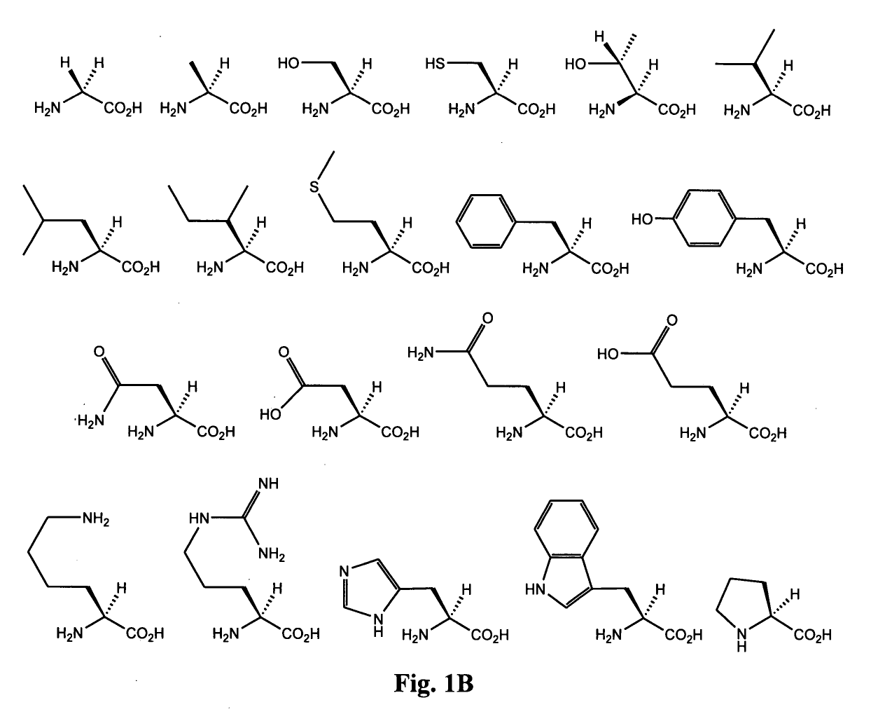

Structural representations of the R groups described herein are shown in FIG. 1A. The filled circle “●” in each of these R groups indicates the carbon atom that is linked to a member of the heterocyclic ring via a single covalent bond. The naturally occurring amino acids are shown in FIG. 1B. FIG. 1B shows the chirality of the natural amino acids that may be employed herein.

The term “cyclic peptide”, as used herein, refers to a heterocyclic compound that is composed of naturally-occurring amino acids that are covalently linked to one another by peptide bonds, where a peptide bond is —(C═O)—(N—H)—. In general, such heterocyclic compounds are a covalently closed circle, and thus are not “loop structures”, such as may be formed by formation of a disulfide bond between cysteines in a polypeptide having more than 3 or 4 residues.

The term “naturally-occurring amino acid” refers to any of the 20 genetically-encodable L isomer amino acids (see, e.g., pages 117-119, “Principles of Biochemistry, Third Ed. Lehninger ed. (2000) Worth Publishers, NY). The 20 naturally-occurring amino acids are alanine, cysteine, aspartic acid, glutamic acid, phenylalanine, glycine, histidine, isoleucine, lysine, leucine, methionine, asparagine, proline, glutamine, arginine, serine, threonine, valine, tryptophan and tyrosine. Reference to “genetically encodable” herein is not meant to be limiting as to the method by which the compound is made, but rather is a convenient reference as to the structure of the amino acid residues. Accordingly, heterocyclic compounds of the invention may be made in a cell (e.g., using an intein system) or synthetically (e.g., using a synthesizer or other chemical means).

For convenience, amino acids are referred to herein by standard one- or three-letter symbols (see, e.g., pages 118, “Principles of Biochemistry, Third Ed. Lehninger ed. (2000) Worth Publishers, NY). In presenting the structure of a heterocyclic compound of the invention using a linear string of one- or three-letter symbols that denote amino acids, it is understood that the first and last amino acids of the string are covalently joined together. Since such a molecule is circular, a cyclic compound may be represented as an amino acid sequence which can be written starting at any point of the sequence. For example, a cyclic peptide having the amino acid sequence “SAW” is identical to a cyclic peptide having the sequence “AWS” or “WSA,” but not the same as the structures “SWA,” “ASW” and “WAS”.

The order of the amino acids in a cyclic peptide follows the N—C convention of linear peptides, where the order of amino acids in a cyclic peptide is described in order from N to C around the cyclic peptide. In other words, cyclo[X₁X₂X₃] describes a cyclic peptide containing three consecutive amino acids X₁, X₂ and X₃, that linked to each other such that their backbones are oriented in the following direction: —N—C—(C═O)—. In certain embodiments, a heterocyclic compound composed of three or four amino acids covalently bound by peptide bonds may be referred to as having the formula cyclo[X₁X₂X₃], or cyclo[X₁X₂X₃X₄], respectively, or where X is an amino acid (e.g., cyclo[SAW]).

The terms “polypeptide” and “protein” are used interchangeably throughout the application and mean at least two covalently attached amino acids.

“Isolated” means that the recited material is unaccompanied by at least some of the material with which it is normally associated when it is first produced (e.g., in a cell). An isolated peptide constitutes at least about 0.1%, at least about 0.5%, at least about 1% or at least about 5% by weight of the total protein in a given sample.

A peptide that is “purified” is a compound that is at least about 50% (e.g., at least about 70% or at least about 90%) pure, by weight, excluding any solvent in which the peptide may be dissolved.

By “inhibitory”, as in the context of an “IRES-inhibitory”, is meant having an activity that inhibits an activity, e.g., IRES mediated translation (i.e., rate of translation initiation by a viral or non-viral IRES). An inhibitory compound generally reduces an activity by at least 20%, e.g., at least 30%, at least 40%, at least 50%, at least 60%, at least 70%, at least 80%, at least 90%, at least 95%, up to about 99% or 100% in an assay, as compared to the same assay performed in the absence of the compound. In general, compounds of interest are those which exhibit IC₅₀s in a particular assay in the range of about 1 mM or less. Compounds which exhibit lower IC₅₀S, for example, in the range of about 100 μM, 10 μM, 1 μM, 100 nM, 10 nM, 1 nM, or even lower, are particularly useful for as therapeutics or prophylactics to treat or prevent a condition, e.g., HCV infections. Alternatively, active compounds are those which exhibit an LD₅₀ (i.e., concentration of compound that reduces viral titer by 50%) in the range of about 1 mM or less. Compounds which exhibit a lower LD₅₀, for example, in the range of about 100 μM, 10 μM, 1 μM, 100 nM, 10 nM, 1 nM, or even lower, are particularly useful for as therapeutics or prophylactics to treat or prevent any condition, for example, HCV infections.

The terms “treat,” “treating,” “treatment,” and the like are used interchangeably herein and mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed the disease such as enhancing the effect of a viral infection. “Treating” as used herein covers treating a disease in a vertebrate and particularly a mammal and most particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

The term “effective amount,” “therapeutic amount,” “therapeutically effective amount,” and the like are used interchangeably here to describe an amount sufficient to effect a treatment, e.g. a beneficial or desired clinical results. An effective amount can be administered in one or more administrations.

Other definitions of terms appear throughout the specification.

### DETAILED DESCRIPTION OF THE INVENTION

As noted above, the present invention provides heterocyclic compounds that exhibit IRES-inhibitory activity.

The IRES-inhibitory heterocyclic compounds may be generally described by the formula: 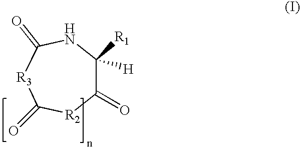
wherein n is 1 or 2, and
wherein R₁ is hydroxymethyl, 1-hydroxyethyl or thiomethyl; and
wherein R₂ and R₃ are, independently:

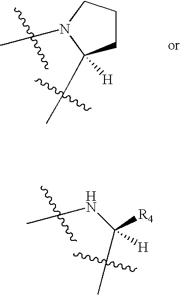

wherein R₄ is hydrogen, methyl, iso-propyl, iso-butyl, sec-butyl, methylthioethyl, benzyl, CH₂-linked 4-hydroxy-phenyl, CH₂-linked indole, hydroxymethyl, thiomethyl, ethanoic amide, propanoic amide, ethanoic acid, propanoic acid, 1-hydroxyethyl, 4-aminobutanyl, 4-(aminoiminomethyl)aminopropyl, hydroxymethyl, 1-hydroxyethyl, thiomethyl or CH₂-linked imidazole.

In certain embodiments,

n is 2,

R₂ is:

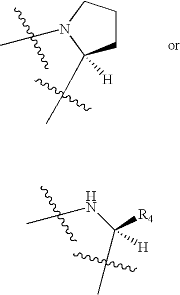

R₄ is hydrogen, methyl, iso-propyl, iso-butyl, sec-butyl, methylthioethyl, benzyl, CH₂-linked 4-hydroxy-phenyl, CH₂-linked indole, hydroxymethyl, thiomethyl, ethanoic amide, propanoic amide, ethanoic acid, propanoic acid, 1-hydroxyethyl, 4-aminobutanyl, 4-(aminoiminomethyl)aminopropyl, hydroxymethyl, 1-hydroxyethyl, thiomethyl or CH₂-linked imidazole and

R₃ is:

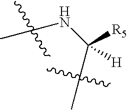

and R₅ is iso-propyl, sec-butyl, methylthioethyl, benzyl, CH₂-linked 4-hydroxy-phenyl, CH₂-linked indole, propanoic amide or 4-aminobutanyl.

In particular embodiments,

n is 2,

R₂ is:

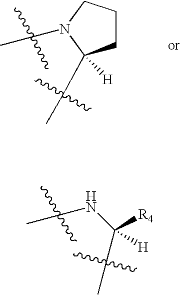

R₄ is hydrogen, methyl, iso-propyl, iso-butyl, sec-butyl, methylthioethyl, benzyl, CH₂-linked 4-hydroxy-phenyl, CH₂-linked indole, hydroxymethyl, thiomethyl, ethanoic amide, propanoic amide, 1-hydroxyethyl, 4-aminobutanyl, 4-(aminoiminomethyl)aminopropyl, hydroxymethyl, 1-hydroxyethyl, thiomethyl or CH₂-linked imidazole and

R₃ is:

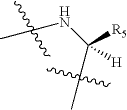

and R₅ is iso-propyl or CH₂-linked indole.

In certain embodiments, the heterocyclic compounds of the invention do not include the following compounds (as represented by their unique CAS registry numbers): 3-mers: 209353-30-0 and 748142-25-8, and 4-mers: 591781-32-7, 189179-32-6, 189179-28-0, 176703-10-9, 176703-09-6, 122886-11-7, 107208-67-3, 83797-39-1, 81017-86-9, 77782-99-1, 135432-38-1, 209353-31-1, and 189179-39-3.

With the exception of hydrogen, the molecular structures of the groups that may be present as R₁, R₄ and R₅ in a subject cyclic compound are shown in FIG. 1A. In each group shown in FIG. 1A, the carbon atom indicated by the filled circle “●” indicate the carbon atom that is linked to a member of the 9-membered heterocyclic ring via a single covalent bond.

In certain embodiments the invention further provides a heterocyclic compound composed of three or four amino acids directly linked to each other by peptide bonds. In the first amino acid position of the heterocyclic compound (corresponding to the amino acid that provides the R₁ group described above), the amino acid may be serine (providing a hydroxymethyl group), threonine (providing a 1-hydroxyethyl group) or cysteine (providing a thiomethyl group). This first amino acid residue is also represented by X₁ in the formula cyclo[X₁X₂X₃X₄], wherein X₄ may be present or absent.

In certain embodiments the invention provides a cyclic compound composed of three or four naturally-occurring amino acids directly linked to each other by peptide bonds. In the first amino acid position of the heterocyclic compound (corresponding to the amino acid that provides the R₁ group described above), the amino acid may be serine (providing a hydroxymethyl group), threonine (providing a 1-hydroxyethyl group) or cysteine (providing a thiomethyl group). This first amino acid residue is also represented by X₁ in the formula cyclo[X₁X₂X₃X₄], wherein X₄ may be present or absent.

In the case of a compound composed of three amino acids, in the second amino acid position of the cyclic compound (corresponding to the amino acids that provides the R₂ group of Formula I), the amino acid may be any amino acid, including glycine (providing a hydrogen group), alanine (providing a methyl group), valine (providing an iso-propyl group), leucine (providing an iso-butyl group), isoleucine (providing a sec-butyl group), methionine (providing a methylthioethyl group), phenylalanine (providing a benzyl group), tyrosine (providing a CH₂-linked 4-hydroxy-phenyl group), tryptophan (providing a CH₂-linked indole group), asparagine (providing an ethanoic amide group), glutamine (providing a propanoic amide group), aspartic acid (providing an ethanoic acid group), glutamic acid (providing a propanoic acid group), lysine (providing a 4-aminobutanyl group), arginine (providing a 4-(aminoiminomethyl)aminopropyl group), histidine (providing a CH₂-linked imidazole group), proline (proving a straight chain C₃H₆ linker between a C and adjoining N of the amino acid), serine (providing a hydroxymethyl group), threonine (providing a 1-hydroxyethyl group) or cysteine (providing a thiomethyl group). In certain embodiments, the amino acid at the second amino acid may be any amino acid that is not acidic (i.e., no negatively charged). This second amino acid residue is also represented by X₂ in the formula cyclo[X₁X₂X₃X₄], wherein X₄ may be present or absent.

In certain embodiments, the third amino acid of a subject compound composed of three amino acids may be phenylalanine, isoleucine, lysine, methionine, glutamine, threonine, tyrosine, valine or tryptophan. In certain embodiments, the amino acid at the third position of a subject compound may be valine or tryptophan. This third amino acid residue is also represented by X₃ in the formula cyclo[X₁X₂X₃X₄], wherein X₄ may be present or absent.

In certain embodiments therefore, the subject compounds may described as being heterocyclic compound of the formula: cyclo[X₁X₂X₃X₄], wherein X₄ may be present or absent, wherein X₁ is a naturally occurring Ser, Thr or Cys amino acid or, in certain embodiments, a non-naturally occurring amino acid selected according to Table 1, X₂, X₃, and X₄ are any naturally-occurring amino acids or a non-naturally occurring amino acid selected according to Table 1, and wherein the amino acids of the compound are joined by peptide bonds. In certain embodiments, the heterocyclic compound is of the formula cyclo[X₁X₂X₃] where X₁ is Ser, Thr or Cys or a non-naturally occurring amino acid selected according to Table 1, X₂ is any naturally-occurring amino acid or a non-naturally occurring amino acid selected according to Table 1, and X₃ is Phe, Ile, Lys, Met, Gln, Tyr, Val or Trp or a non-naturally occurring amino acid selected according to Table 1. In particular embodiments, the heterocyclic compound is of the formula cyclo[X₁X₂X₃], where X₁ is Ser, Thr or Cys or a non-naturally occurring amino acid selected according to Table 1, X₂ is any naturally-occurring amino acid that is not Asp or Glu or a non-naturally occurring amino acid selected according to Table 1, and X₃ is Val or Trp or a non-naturally occurring amino acid selected according to Table 1. The amino acid at X₂ may be aromatic, apolar, aliphatic, basic or polar, for example.

In particular embodiments, the IRES-inhibitory heterocyclic compound of the invention comprise an amino acid sequence selected from any of the following cyclic amino acid sequences: cyclo[CAW], cyclo[CMW], cyclo[CWW], cyclo[CYW], cyclo[SAW], cyclo[SFV], cyclo[SFW], cyclo[SIV], cyclo[SIW], cyclo[SKV], cyclo[SLW], cyclo[SMV], cyclo[SMW], cyclo[SVI], cyclo[SVV], cyclo[SVW], cyclo[SWF], cyclo[SWI], cyclo[SWM], cyclo[SWV], cyclo[SWW], cyclo[SWY], cyclo[SYV], cyclo[SYW], cyclo[TCW], cyclo[TFW], cyclo[THW], cyclo[TLW], cyclo[TMF], cyclo[TMV], cyclo[TMW], cyclo[TQW], cyclo[TSW], cyclo[TYW], cyclo[SAV], cyclo[SDV], cyclo[SEV], cyclo[SHV], cyclo[STV], cyclo[SAM], cyclo[SAT], cyclo[SAY], cyclo[TAV], cyclo[SAF], cyclo[SAI], cyclo[SAK], cyclo[SAQ], cyclo[SHW], cyclo[SPW], cyclo[SQW], cyclo[SRW], cyclo[STW], cyclo[SNW], cyclo[SSW], cyclo[SCW], cyclo[SEW], cyclo[SGW], cyclo[SDW], cyclo[TAW], cyclo[SWFR], cyclo[SWFA], cyclo[SWFK], cyclo[SWFM], cyclo[SWWR], cyclo[SWYR], cyclo[TWFR] and cyclo[SFWR]. The amino acids of the above compounds may be naturally occurring, or non-naturally occurring amino acid and selected according to Table 1. The structures of the above compounds are shown in Table 3 below.

TABLE 3

| Entry | Name | Structure |
|---|---|---|
| 1 | c[SAW] | 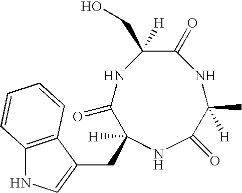 |
| 2 | c[SWW] | 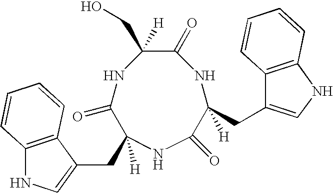 |
| 3 | c[SAV] | 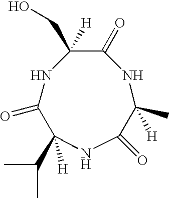 |
| 4 | c[SVV] | 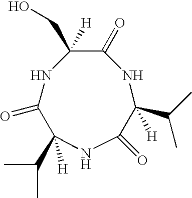 |
| 5 | c[SIV] | 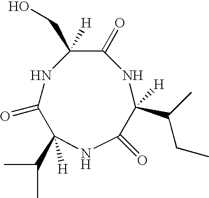 |
| 6 | c[TYW] | 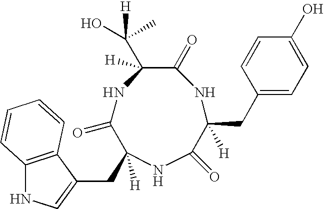 |
| 7 | c[TMW] | 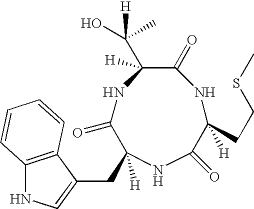 |
| 8 | c[SAF] | 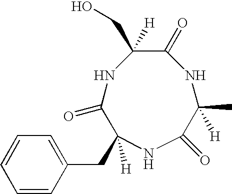 |
| 9 | c[SAI] | 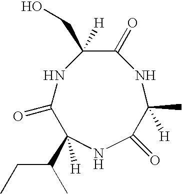 |
| 10 | c[SAK] | 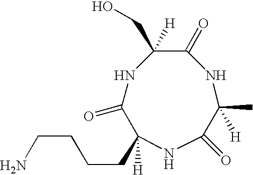 |
| 11 | c[SAM] | 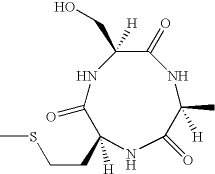 |
| 12 | c[SAQ] | 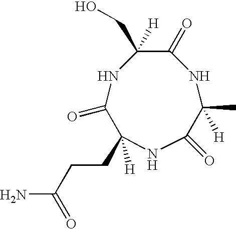 |
| 13 | c[SAT] | 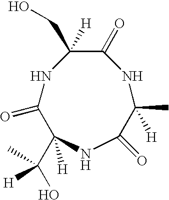 |
| 14 | c[SAY] | 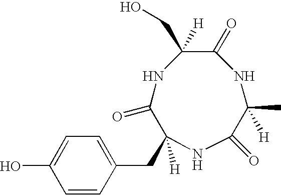 |
| 15 | c[SWY] | 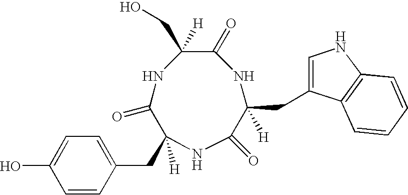 |
| 16 | c[SCW] | 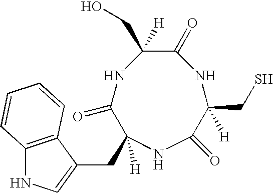 |
| 17 | c[SDW] | 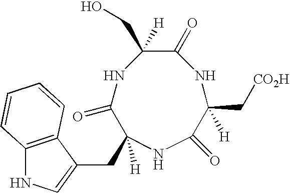 |
| 18 | c[SEW] | 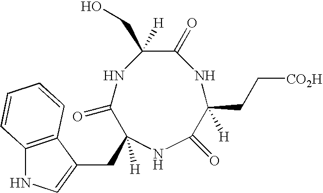 |
| 19 | c[SFW] | 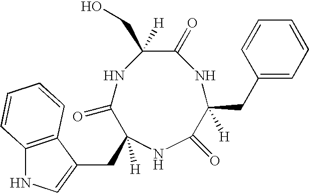 |
| 20 | c[SGW] | 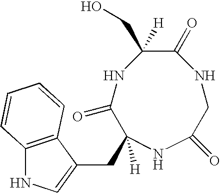 |
| 21 | c[SHW] | 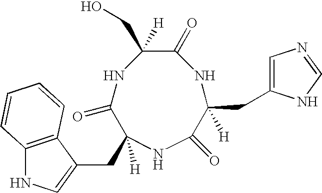 |
| 22 | c[SIW] | 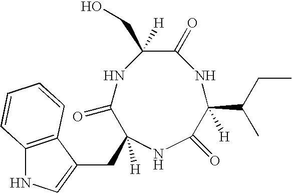 |
| 23 | c[SMW] | 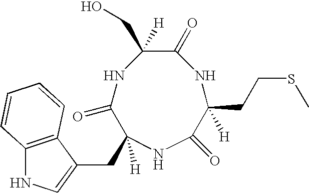 |
| 24 | c[SNW] | 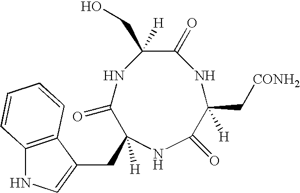 |
| 25 | c[SPW] | 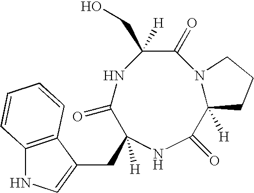 |
| 26 | c[SQW] | 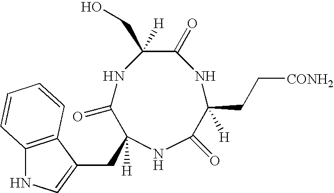 |
| 27 | c[SRW] | 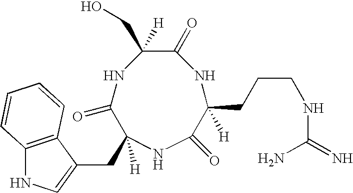 |
| 28 | c[SSW] | 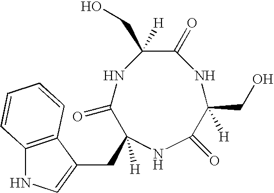 |
| 29 | c[STW] | 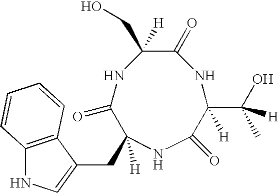 |
| 30 | c[SVW] | 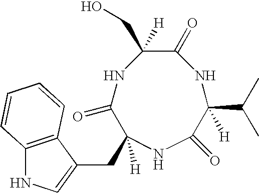 |
| 31 | c[SYW] | 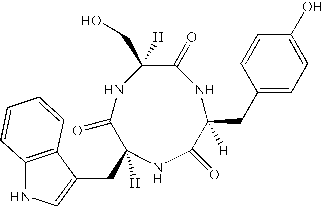 |
| 32 | c[SDV] | 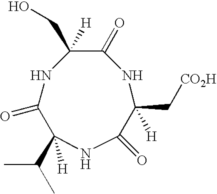 |
| 33 | c[SEV] | 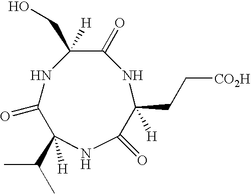 |
| 34 | c[SFV] | 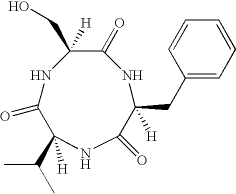 |
| 35 | c[SHV] | 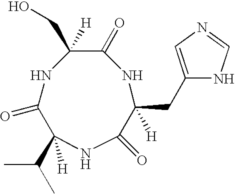 |
| 36 | c[SKV] | 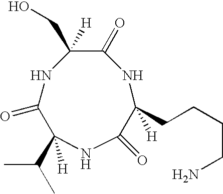 |
| 37 | c[SLW] | 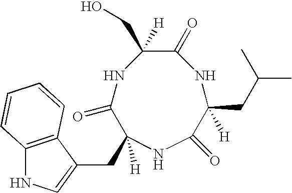 |
| 38 | c[SMV] | 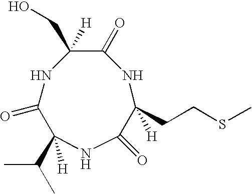 |
| 39 | c[SYV] | 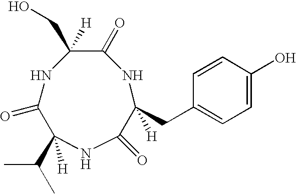 |
| 40 | c[SWV] | 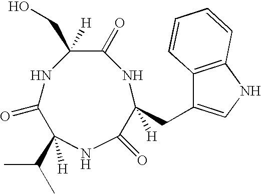 |
| 41 | c[STV] | 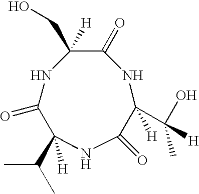 |
| 42 | c[SVI] | 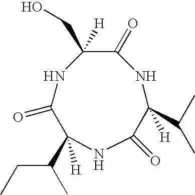 |
| 43 | c[SWF] | 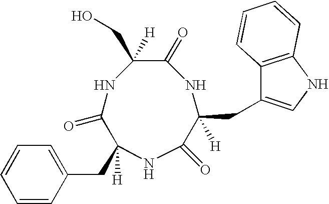 |
| 44 | c[SWI] | 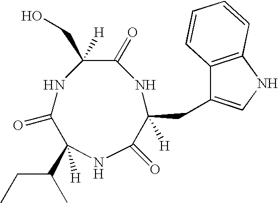 |
| 45 | c[SWM] | 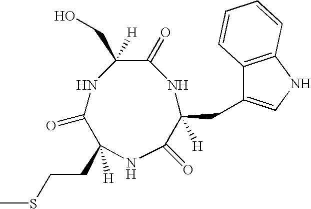 |
| 46 | c[TAV] | 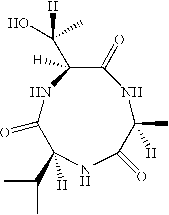 |
| 47 | c[TAW] | 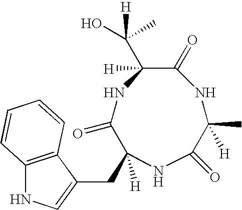 |
| 48 | c[TFW] | 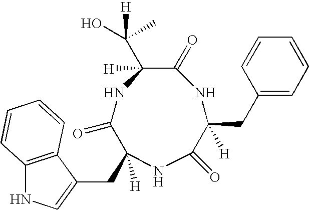 |
| 49 | c[THW] | 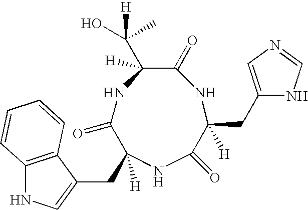 |
| 50 | c[TSW] | 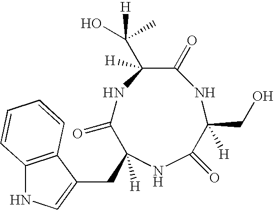 |
| 51 | c[TMV] | 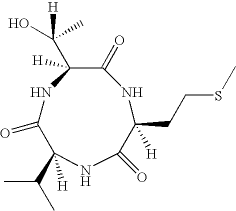 |
| 52 | c[TMF] | 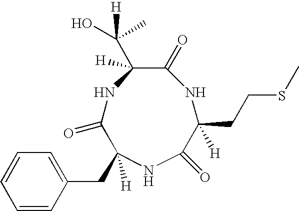 |
| 53 | c[CWW] | 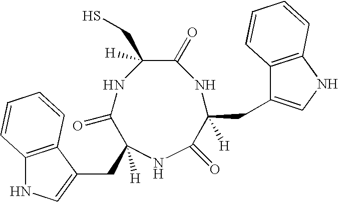 |
| 54 | c[CYW] | 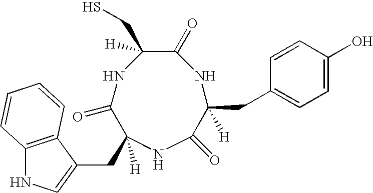 |
| 55 | c[CMW] | 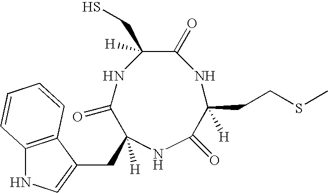 |
| 56 | c[CAW] | 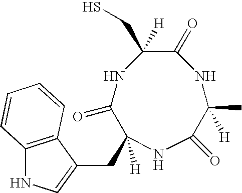 |
| 57 | c[TCW] | 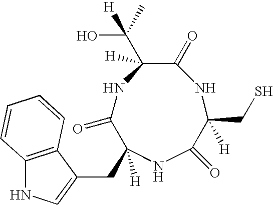 |
| 58 | c[TLW] | 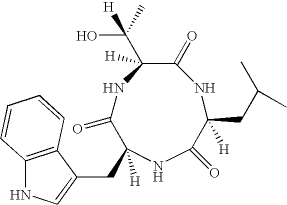 |
| 59 | c[TQW] | 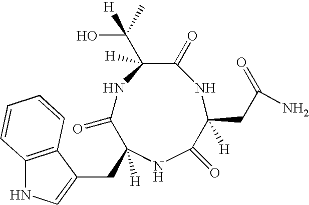 |
| 60 | c[SWFR] | 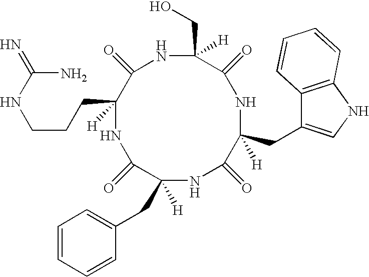 |
| 61 | c[SWFA] | 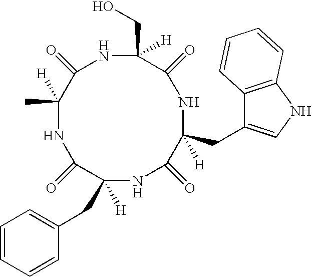 |
| 62 | c[SWFK] | 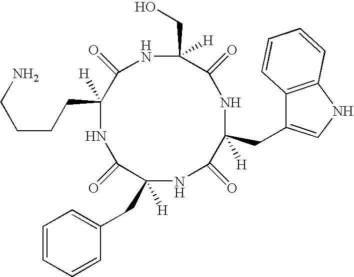 |
| 63 | c[SWFM] | 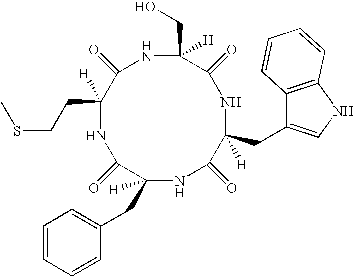 |
| 64 | c[SWWR] | 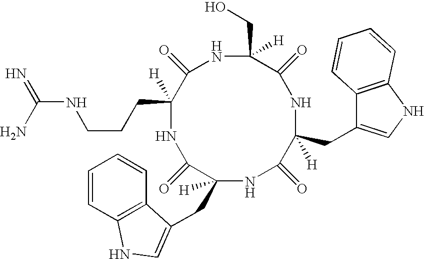 |
| 65 | c[SWYR] | 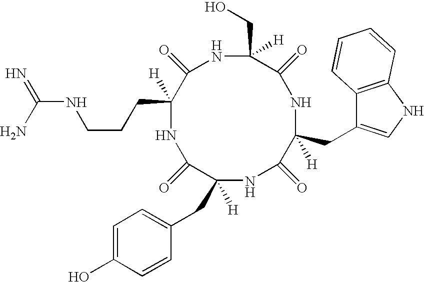 |
| 66 | c[TWFR] | 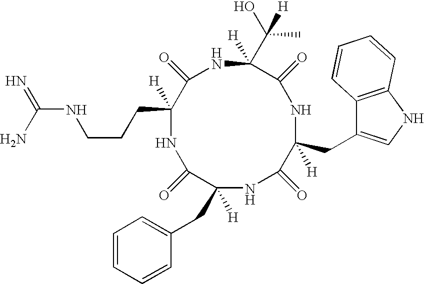 |
| 67 | c[SFWR] | 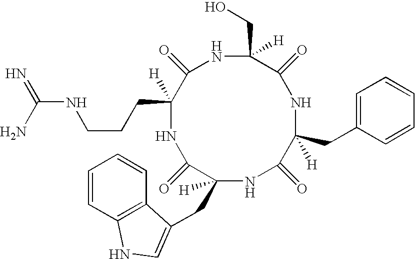 |

The IRES-inhibitory compounds of the invention were discovered in a cellular screening assay that involves producing a library of cyclic peptides in mammalian cells using an intein system (similar to that described by Kinsella et al. J Biol. Chem. 2002 277:37512-8), and determining whether those cyclic peptides decrease expression of an IRES-regulated reporter protein.

The IRES-inhibitory compounds of the invention are IRES-specific as determined by assays designed to identify compounds that inhibit IRES-mediated translation, but not significantly inhibit expression of a reporter linked to a 5′ capped cellular untranslated region (UTR), e.g., particularly a mammalian UTR such as a human UTR. The subject IRES inhibitory compounds therefore inhibit protein translation of IRES-containing viruses, but do not inhibit translation of cellular-encoded proteins from capped cellular mRNAs. Accordingly, the compounds of the invention may be employed to inhibit replication of those viruses that contain an IRES. Such IRES-containing viruses include, but are not limited to: picornaviruses (e.g., polioviruses, rhinoviruses, coxsackie viruses), HIV, hepatitis A virus, foot-and-mouth disease viruses, and Flaviviridae viruses (i.e., viruses belonging to the Flaviviridae family, e.g., flaviviruses, pestiviruses and hepaciviruses, including, yellow fever virus (YFV); Dengue virus, including Dengue types 1-4; Japanese Encephalitis virus; Murray Valley Encephalitis virus; St. Louis Encephalitis virus; West Nile virus; tick-borne encephalitis virus; Hepatitis C virus (HCV); Kunjin virus; Central European encephalitis virus; Russian spring-summer encephalitis virus; Powassan virus; Kyasanur Forest disease virus; and Omsk hemorrhagic fever virus).

HCV is of particular interest in the invention. The HCV contemplated by the invention may be of any genotype (e.g., genotype 1, 2, 3, 4, 5, 6, or the like), as well as subtypes of an HCV genotype (e.g., 1a, 1b, 2a, 2b, 3a, etc.). Because currently HCV genotype 1 is normally the most difficult to treat, HCV genotype 1 and genotype 1 subtypes are of particular interest. Of further related interest is treatment of patients who have failed HCV therapy, e.g., IFN-α monotherapy, IFN-α combination therapy, and the like. Treatment failure patients include patients who never significantly respond to therapy (“nonresponders”) as well as patients who initially respond and then relapse (“relapsers”).

While the specification below may specifically refer to HCV, such a reference is only for clarity and is not intended to limit the invention to use in the context of HCV as described in more detail below. As noted above, the invention can be applied to any virus encoding an IRES such as any Flaviviridae virus or a picornavirus, for example.

In addition to IRES-inhibitory heterocyclic compounds that contain naturally-occurring amino acids, IRES-inhibitory heterocyclic compounds that contain non-naturally occurring amino acids are also contemplated, as well as heterocyclic compounds containing amino acid substitutions (e.g., conservative amino acid substitutions).

The IRES-inhibitory heterocyclic compounds that contain non-natural amino acids have substantially the same structural and/or functional characteristics of the heterocyclic compounds set forth above. A subject IRES-inhibitory heterocyclic compound containing non-natural amino acids can be entirely composed of synthetic, non-natural analogues of natural amino acids, or can be a chimeric molecule containing both natural and non-natural amino acids. A subject IRES-inhibitory heterocyclic compounds may also incorporate any amount of natural amino acid substitutions (particularly conservative amino acid substitutions) as long as such substitutions also do not substantially alter the peptide's IRES inhibitory activity.

Routine experimentation is all that is required to determine if a particular IRES-inhibitory heterocyclic compound is within the scope of the invention.

Subject IRES-inhibitory heterocyclic compound containing non-natural amino acids generally contain any combination of non-natural components, including: a) residue linkages other than natural amide bonds (“peptide bonds”); and/or b) non-natural amino acid residues in place of natural amino acid residues.

Amino acid residue linkages other than amide bonds (i.e., —C(═O)—NH—) that may be employed in the subject compositions include, but are not limited to: ketomethylene bonds (e.g., —C(═O)—CH₂—), aminomethylene bonds (CH₂—NH), ethylene bonds (—C₂H₄—), olefin bonds (—CH═CH—), ether bonds (—CH₂—O—), thioether bonds (—CH₂—S—), tetrazole bonds (CN₄—), as well as thiazole, retroamide, thioamide, and ester bonds. Such “surrogate” peptide bonds are well known in the art (see, e.g., Spatola (1983) in Chemistry and Biochemistry of Amino Acids, Peptides and Proteins, Vol. 7, pp 267-357, A Peptide Backbone Modifications, Marcell Dekker, NY) and are readily employed herein. For ease of description, such linkages may still termed “peptide” or “amino” bonds herein, although the linkage may not have a conventional “peptide” or “amino” bonds structure: —C(═O)—NH—

Further, as mentioned above, amino acids of the exemplary IRES-inhibitory heterocyclic compounds discussed above may be replaced by either: a) non-natural amino acids or b) different natural amino acids, as long as the replacing amino acids have similar properties (based on size, polarity, hydrophobicity, and the like) to the amino acid to be replaced. In other words, any of the natural amino acids of any of the heterocyclic compounds shown listed above may be replaced by a different natural amino acid or a non-natural amino acid of the same class, where natural and exemplary non-natural amino acids are classified according to Table 1. In certain cases, certain positions of a subject heterocyclic compound may not be essential for activity of the heterocyclic compound. In these cases, the inessential amino acids may be substituted with other amino acids or linker moieties that improve the biochemical properties (e.g., solubility or permeability, etc.) of the heterocyclic compound or increase potency.

As noted above, certain compounds of the invention may contain one or more (e.g., one, two or three) non-natural amino acids. It is recognized that the amino acid side chains of compounds of Formula I may be the side chains of naturally occurring amino acids (as set forth in FIG. 1B). In certain embodiments, an amino acid of a cyclic peptide of the invention may contain the side chain of one or more of the non-naturally occurring amino acids listed in Table 1. For example, R₄ or R₅ may be the side-chain of norleucine, which, as is well known in the art, is a butanyl residue. The structures of the amino acids listed in Table 1 are well known.

TABLE 1

| Classification | Natural | Non-natural |
|---|---|---|
| Aromatic | F, Y, W | Phg, Nal, Thi, Tic, Phe(4-Cl), |
| | | Phe(2-F), Phe(3-F), Phe(4-F), |
| | | Pyridyl Ala, Benzothienyl Ala |
| Apolar | L, V, I, A, M | T-BuA, T-BuG, MeIRe, Nle, |
| | | MeVal, G, P Cha, MeGly, Aib |
| Aliphatic | A, V, L, I | t-BuA, t-BuG, MeIle, Nle, MeVal, |
| | | Cha, bAla, MeGly, Aib, Dpr, Aha |
| Acidic | D, E | |
| Basic | H, K, R | Dpr, Orn, hArg, Phe(p-NH₂), Dbu, |
| | | Dab |
| Polar | Q, N, S, T, Y | Cit, AcLys, MSO, hSer, bAla |
| Cysteine-Like | C | Pen, hCys, p-methyl Cys |

The non-natural amino acids of Table 1 are abbreviated as follows; β-alanine (β-Ala) and other amino acids such as 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,4-diaminobutyric acid (Dbu); p-aminophenylalanine (Phe(pNH.sub.2)); N-methyl valine (MeVal); homocysteine (hCys) and homoserine (hSer).

Each class of amino acids set forth in Table 1 is discussed in greater detail below.

A hydrophobic amino acid is an amino acid exhibiting a hydrophobicity of greater than zero according to the normalized consensus hydrophobicity scale of Eisenberg et al. J. Mol. Biol. 179: 125-142). Examples of natural hydrophobic amino acids include Pro, Phe, Trp, Met, Ala, Gly, Tyr, Ile, Leu and Val. Examples of non-natural hydrophobic amino acids include t-BuA.

An aromatic amino acid is a hydrophobic amino acid having a side chain containing at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Examples of natural aromatic amino acids include Phe, Tyr and Trp. Commonly encountered non-natural encoded aromatic amino acids include phenylglycine, 2-naphthylalanine, □-2-thienylalanine, 1,2,3,4-tetrahydroisoquinolin-e-3-carboxylic acid, 4-chloro-phenylalanine, 2-fluorophenylalanine, 3-fluorophenylalanine and 4-fluorophenylalanine. Aromatic rings of a non-natural amino acid include, thiazolyl, thiophenyl, pyrazolyl, benzimidazolyl, naphthyl, furanyl, pyrrolyl, and pyridyl aromatic rings.

An apolar amino acid is a hydrophobic amino acid having a side chain that is uncharged at physiological pH and which has bonds in which the pair of electrons shared in common by two atoms is generally held equally by each of the two atoms (i.e., the side chain is not polar). Examples of natural apolar amino acids include Gly, Leu, Val, Ile, Ala and Met. Examples of non-natural apolar amino acids include Cha.

An aliphatic amino acid is a hydrophobic amino acid having an aliphatic hydrocarbon side chain. Examples of natural aliphatic amino acids include Ala, Leu, Val and Ile. Examples of non-natural aliphatic amino acids include Nle.

A hydrophilic amino acid is an amino acid exhibiting a hydrophilicity of less than zero according to the normalized consensus hydrophobicity scale of Eisenberg et al. J. Mol. Biol. 179: 125-142). Examples of natural hydrophilic amino acids include Thr, His, Glu, Asn, Gln, Asp, Arg, Ser and Lys. Examples of natural hydrophilic amino acids include Cet and hCys.

An acidic amino acid is a hydrophilic amino acid having a side chain pK value of less than 7. Acidic amino acids typically have negatively charged side chains at physiological pH due to loss of a hydrogen ion. Examples of natural acidic amino acids include Asp and Glu.

A basic amino acid is a hydrophilic amino acid having a side chain pK value of greater than 7. Basic amino acids typically have positively charged side chains at physiological pH due to association with hydronium ion. Examples of natural basic amino acids include Arg, Lys and His. Examples of non-natural basic amino acids include the non-cyclic amino acids ornithine, 2,3-diaminopropionic acid, 2,4-diaminobutyric acid and homoarginine.

A polar amino acid is a hydrophilic amino acid having a side chain that is uncharged at physiological pH, but which has one bond in which the pair of electrons shared in common by two atoms is held more closely by one of the atoms. Examples of natural polar amino acids include Ser, Thr, Asn and Gln. Examples of non-natural polar amino acids include citrulline, N-acetyl lysine and methionine sulfoxide.

The amino acid residue Cys has the ability to form disulfide bridges with other Cys residues or other sulfanyl-containing amino acids. Cys is classified as a polar hydrophilic amino acid for the purposes of the present invention. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

Any particular residue of a subject heterocyclic compound can also be replaced by an amino acid of the opposite chirality. Thus, any amino acid naturally occurring in the L-configuration can be replaced with the amino acid of the same or similar chemical structure, but of the opposite chirality, generally referred to as the D-amino acid.

As would be recognized by one of skill in the art, conservative amino acid substitutions could be made in a subject cyclic polypeptide without altering the IRES-inhibiting activity of that polypeptide. Table 2 illustrates exemplary amino acid substitutions that may be made:

TABLE 2

| Amino acid to | Replacing |
|---|---|
| be replaced | amino acid |
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In certain embodiments, an amino acid of a subject heterocyclic compound may be replaced by an organic liker that preserves the spacing of the replaced amino acid.

The subject IRES-inhibitory heterocyclic compounds may be made in a cell using well known intein-based methods, for example. For example, U.S. patent application 20040014100, Camarero and Muir (J. Am. Chem. Soc. 1999 121:5597-5598), Iwai and Pluckthun (FEBS Lett. 1999 459:166-172), Evans, et al. (J. Biol. Chem. 1999 274:18359-18363); Scott et al. (Proc. Natl. Acad. Sci. 1999 96:13638-13643) and Kinsella et al. (J. Biol. Chem. 2002 277:37512-8) each describe intein based methods in which subject cyclic polypeptides may be made in a cell, and are incorporated by reference herein in their entireties.

Alternatively, a subject IRES-inhibitory heterocyclic compound may be made synthetically using standard chemical synthesis, for example, by the solid phase peptide synthesis method of Merrifield et al. (J. Am. Chem. Soc. 1964 85:2149). Standard solution methods may also be used (see, for example, Bodanszky, Principles of Peptide Synthesis, Springer-Verlag, Berlin (1984) and Bodanszky, Peptide Chemistry, Springer-Verlag, Berlin (1993)). Subject biopolymers can be chemically synthesized by the methods of Creighton (1983, Proteins: Structures and Molecular Principles, W.H. Freeman & Co., N.Y) or Hunkapiller et al. (Nature, 310:105-111 (1984)). Once produced, a linear peptide can be cyclized using known chemistry.

In certain embodiments, the subject IRES-inhibitory heterocyclic compound may be made using semi-synthetic means in which a linear peptide is made synthetically, ligated to two domains of an intein, and cyclized in a cell-free reaction. These methods are described in U.S. provisional patent application 60/574,238, filed on May 24, 2004, entitled “METHODS FOR CYCLIZING SYNTHETIC POLYMERS”, which application is incorporated herein in its entirety.

Formulations and Routes of Administration

The compounds described herein can be formulated in a variety of ways suitable for administration. In general, these compounds are provided in the same or separate formulations in combination with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the agents are formulated separately or in combination, e.g., in an aqueous or non-aqueous formulation, which may further include a buffer. Suitable aqueous buffers include, but are not limited to, acetate, succinate, citrate, and phosphate buffers varying in strength from 5 mM to 100 mM. In some embodiments, the aqueous buffer includes reagents that provide for an isotonic solution. Such reagents include, but are not limited to, sodium chloride, and sugars e.g., mannitol, dextrose, sucrose, and the like. In some embodiments, the aqueous buffer further includes a non-ionic surfactant such as polysorbate 20 or 80.

Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In the subject methods, the active agents may be administered to the host using any convenient means capable of resulting in the desired therapeutic effect. Thus, the agents can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, agents may be administered in the form of their pharmaceutically acceptable salts, or they may also be used alone or in appropriate association, as well as in combination, with other pharmaceutically active compounds. The following methods and excipients are merely exemplary and are in no way limiting.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature. Agents can also be provided in sustained release or controlled release formulations, e.g., to provide for release of agent over time and in a desired amount (e.g., in an amount effective to provide for a desired therapeutic or otherwise beneficial effect).

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of the agents calculated in an amount sufficient to produce the desired effect in association with a pharmaceutically acceptable diluent, carrier or vehicle. The specifications for the unit dosage forms for use in the present invention depend on the particular compound employed and the effect to be achieved, the pharmacodynamics associated with each compound in the host, and the like.

Dosage forms of particular interest include those suitable to accomplish intravenous or oral administration, as well as dosage forms to provide for delivery by a nasal or pulmonary route (e.g., inhalation), e.g., through use of a metered dose inhaler and the like.

In general, agents for use in the invention is formulated in either parenteral or enteral forms, usually enteral formulations, more particularly oral formulations. Agents for use in the invention are formulated for parenteral administration, e.g., by subcutaneous, intradermal, intraperitoneal, intravenous, or intramuscular injection. Administration may also be accomplished by, for example, enteral, oral, buccal, rectal, transdermal, intratracheal, inhalation (see, e.g., U.S. Pat. No. 5,354,934), etc.

Methods of Use of IRES-Inhibitory Cyclic Compounds

The invention further provides methods for inhibiting viral IRES-mediated translation. These methods generally involve contacting a viral IRES with an IRES-inhibitory heterocyclic compound, as described above, in an amount effective to inhibit translation mediated by the IRES.

The methods may be performed using a cell-free system employing an in vitro translation system employing, e.g., reticulocyte lysate. See, e.g., Jang et al (J. Virol. 1989 63: 1651-1660), Shih (J. Virol. 1979 30:472-80) and Tsukiyama-Kohara et al (J. Virol. 1992 66:1476-83) for details of these methods. Alternatively, the subject methods may be performed using a cell, particularly a mammalian host cell. In these embodiments, the methods generally involve contacting a cell with an above-described heterocyclic compound to inhibit translation initiation from the IRES.

In any of these embodiments, the IRES may be encoded by and produced using naturally occurring viral (e.g., HCV) genome or any man-made replicon thereof. In certain embodiments, however, the IRES may be operably linked to (i.e., initiating the translation of) a polypeptide coding sequence to which it is not naturally linked. For example, the IRES may be operably linked to an optically-detectable reporter protein coding sequence, such as a polynucleotide encoding a light-emitting or color-generating protein.

As mentioned above, in certain embodiments, the methods may be performed in a cellular environment. The cell may be in vitro (e.g., a cultured cell), ex vivo (e.g., in an intact organ removed from a mammalian subject such as a removed liver), or in vivo (e.g., an animal model for a viral infection, e.g, an animal model for HCV, or in a mammalian subject). The IRES may be produced by a wild-type virus, a man-made replicon thereof, or using a recombinant nucleic acid encoding, for example, an IRES-dependent reporter protein. Such nucleic acids may be introduced into a cell using a variety of means, including transfection by a retroviral vector.

Similarly, the invention also provides a method of inhibiting viral replication in a virus-infected cell. In general, these methods involve contacting a cell with a subject heterocyclic compound in an amount effective to inhibit viral replication in the cell.

In general, methods involve contacting a cell infected with an IRES containing virus or a model thereof, e.g., HCV or model thereof (e.g., an HCV subgenomic replicon; Lim, Virology. 2002 303(1):79-99), with an above-described heterocyclic compound, and inhibiting viral replication. Again, the cell may be a cell in vitro, ex vivo or in vivo.

In certain embodiments, the subject methods may involve inhibiting HCV replication in replicon cells. HCV replication assays in which the subject compounds may be employed are described in Lohmann et al (1999 Science 258:110-113), WO03/040112 and WO2004018463. Other embodiments may employ an HCV infection and replication assays, as described in Fournier et al, (1998 J. Gen. Virol. 79:2367-2374).

In general, a heterocyclic compound described above will reduce viral replication by up to about 20%, up to about 30%, up to about 40%, up to 50%, up to about 80%, up to about 90% or up to about 95% or more, using a standard replicon colony formation assay, as compared to controls in the absence of an agent.

In some embodiments of the invention an agent is contacted with a cell that is already infected with the IRES-containing virus, or, in certain other embodiments, an agent is contacted with a cell before its infection with IRES-containing virus. In these embodiments, the subject heterocyclic compound may be administered as a prophylactic, e.g., to increase the viability of a cell and provide “protection” of a cell against a future viral infection or to protect a normal transplanted liver from an IRES-containing virus in a host, for example.

In any of the above methods, the virus or replicon thereof may be any virus containing an IRES, including Flaviviridae viruses, e.g., HCV.

Certain of the above-methods may be performed on a non-human animal model for HCV. Many such animal models using mammals, especially of mouse, monkeys, rats, cats, dogs, guinea pigs, chimpanzees, etc., are known to one of skill in the art. Mouse models, in particular the mouse models for HCV, described in PCT publication WO01/67854, may be used. Other models include those described in WO 99/16307 and Galun et al. J. Infect. Dis. 172:25-30 (1995), describing transplantation of HCV-infected human hepatocytes into liver of immunodeficient mice; Bronowicki et al. Hepatology 28:211-8 (1998), describing intraperitoneal injection of HCV-infected hematopoietic cells into SCID mice; and Lerta et al. Hepatology 28(4Pt2):498A (1998), describing mice transgenic for the HCV genome.

In many embodiments, upon administration of a subject agent, a symptom (e.g. viability of pathogen infected cells, lesions, bleeding, bruising, titer, ALT, the number of infected cells) of the pathogen exhibited by the animal is reduced up to 20%, up to 50%, up to 70%, up to 80%, up to 90%, up to 95%, up to 98%, and even up to 99% or 99.5% as compared to an animal that is not administered a subject agent. In other embodiments, upon administration of a subject agent, a symptom (e.g. CD4 count, ALT or HAAT activity, etc.) of the pathogen exhibited by the animal is increased up to 20%, up to 50%, up to 70%, up to 80%, up to 90%, up to 95%, up to 98%, and even up to 99% or 99.5%, as compared to an animal that is not administered a subject agent.

In many embodiment, a blood sample is taken from the animal and tested for the level of a blood product, such as a virus, cell, a protein, or a molecule (e.g. viral titer, viral genome, viral mRNA, CD4 count or HAAT activity etc.). In other embodiments, a sample of tissue is taken from the test animal and symptoms (e.g. cell death, lesions, viral titer etc) are measured.

Kits

Also provided are reagents and kits thereof for practicing one or more of the above-described methods. The subject reagents and kits thereof may vary greatly. Typically, the kits at least include an IRES-inhibitory heterocyclic compound, as described above. The subject kits may also include one or more additional reagents, e.g., a pharmaceutically acceptable excipient or the like for dissolving the peptide (if it is in dry form, for example) and other reagents for administering the peptide to a mammalian subject, e.g., a human.

In addition to the above components, the subject kits can further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Utility

The invention finds use in the treatment of virus infection in a subject. In particular, the invention finds use in the treatment of an IRES-containing virus (e.g., HCV) infection in a mammalian subject. In this context, treatment can involve reduction of viral load in the infected subject (e.g., reduction of viral load or viral titer). The invention also contemplates preventing or reducing the risk of symptoms or disease of infection by a virus in a susceptible subject. Examples of subjects in this latter category include, but are not necessarily limited to, organ transplant recipients (e.g., liver transplants, bone marrow or other immune cell transplants, and the like). Of particular interest is treatment of a subject having a chronic viral infection, including those undergoing liver transplant as therapy so as to clear the viral infection and reduce the risk of re-infection of the donor liver and immunocompromised or otherwise immune deficient subjects (e.g., due to autoimmune disease, AIDS, genetic defect, and the like).

Specific exemplary intracellular pathogen infections contemplated for treatment according to the invention include, but are not necessarily limited to, those infections associated with hepatitis C virus (HCV, including HCV genotypes 1, 2, 3, and the like).

The virus may be present in a virulent, latent, or attenuated form, or in a combination of those forms. The subject may be symptomatic or asymptomatic.

In one embodiment, a liver that is to be transplanted into a patient is treated with a subject agent prior to transplantation. In another embodiment, the liver of an infected patient is removed from the patient, treated with a subject agent, and placed back into the patient.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The agents of the invention can be administered as a sole active agent, in combination (together or serially) with (i.e., as a “cocktail” with) one or more other medicaments, such as, for example, ribavirin and/or ribavirin derivatives, IFN-α (e.g., IFN-α2a, IFN-α2b, PEG-IFN-α2a, PEG-IFN-α2b, consensus IFN (e.g., INFERGEN™), PEGylated consensus IFN), reverse transcriptase inhibitors (e.g., a dideoxynucleoside including AZT, ddI, ddC, d4T, 3TO, FTC, DAPD, 1592U89 or CS92); and other agents such as 9-(2-hydroxyethoxymethyl) guanine (acyclovir), ganciclovir or penciclovir, interleukin II, or in conjunction with other immune modulation agents including bone marrow or lymphocyte transplants or other medications such as levamisol or thymosin which would increase lymphocyte numbers and/or function as is appropriate.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the subject invention.

### Example 1

### Screening Methods

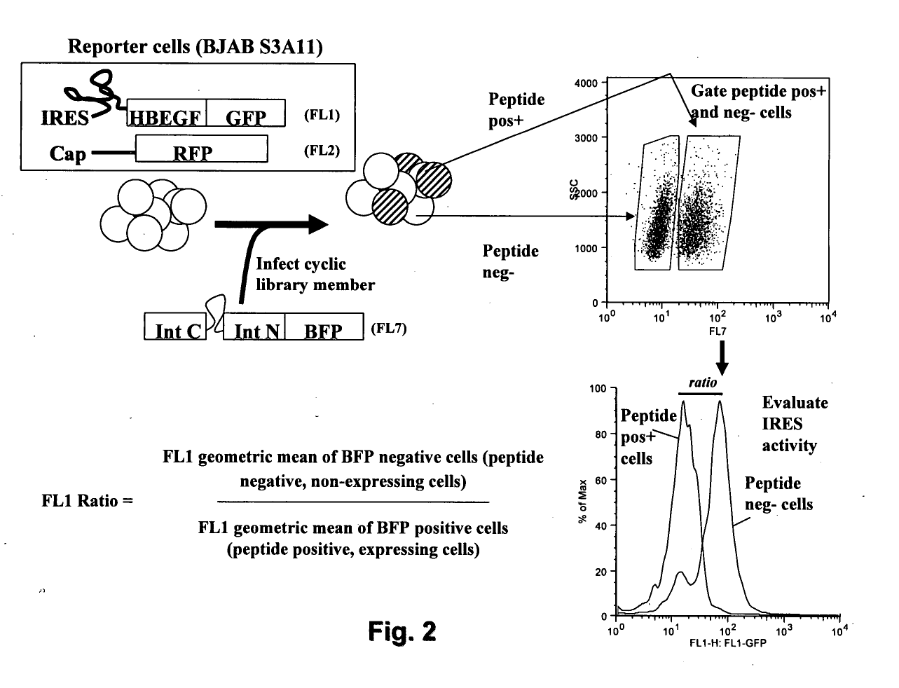

Cells producing libraries of cyclic 3-mers and 4-mers (i.e., libraries of cyclic peptides that contain 3 or 4 amino acid residues) were constructed using methods identical to those of Kinsella et al (J Biol. Chem. 2002 277:37512-8), except different oligonucleotides were used for library construction. Clones from this library were screened in an assay shown in FIG. 2 to identify IRES-inhibitory compounds.

In summary, separate cyclic 3-mer and cyclic 4-mer libraries were constructed such that each peptide of the library contained a fixed Ser, Thr or Cys residue (at position 1) and the remaining residues were randomized. For library construction, degenerate oligonucleotides of sequence 5′-AAGATCATATGACATCATCGTCCACAAC(AGC/ACC/TGC)(NNK)_{2 or 3} TGCATCAGCGGCGACAG-3′ were annealed to the primer 5′-CTTGCCGGTGCTGGCCAGGCTGATCAGGCTGTCGCCGCTGATGCA-3′ and extended using the Expand PCR kit (Roche Molecular Biochemicals). The double-stranded DNA insert was digested and inserted into the BclI/DrdI sites of DnaBO-e-BFP (ACUC). The plasmid libraries were electroporated into ElectroMAX DH 10B competent E. coli (Invitrogen) for amplification on LB+amp agar medium. As illustrated in FIG. 2, the individual library members encode a fusion protein containing a blue fluorescent protein (BFP), the C and N terminal domains of an intein, and a peptide that is to be cyclized. Expression of the intein is detected by detecting BFP expression. Expression of the intein (and the cyclic peptide produced by the intein) is suppressible by addition of exogenous doxycycline. The amino acid sequence of the junctions of the intein scaffold used in making the 3-mer library is Val-His-Asn-X_{3 or 4}-Cys-Ile-Ser, where X_{3 or 4} are three or four contiguous amino acids.

Infectious retroviral particles were produced by transfection of 15 □g of each library (as described in Swift et al, (1999). Current Protocols in Immunology Vol. 10.17C, pp. 1-17, Freeman, N.Y.) into approximately 5×10⁶ Phoenix-A packaging cells. Each resulting library of retroviral particles was then used to infect 5×10⁶ BJAB-S3A11 reporter cells and a FACS based selection was carried out to isolate cells with reduced HCV-IRES reporter output.

In detail, referring to FIG. 2, the cells used in the assay contained an IRES-dependent reporter system. The IRES dependent reporter system contains a CMV promoter that drives the transcription of an RNA containing an HCV IRES, operably linked to a dual function reporter protein-encoding RNA. Translation of the dual function reporter protein is dependent on the activity of the IRES. Expression of the dual function reporter, HBEGF-GFP, leads to expression of both HBEGF (the diphtheria toxin receptor) and GFP (green fluorescent protein). Expression of HBEGF makes the cells sensitive to diphtheria toxin and GFP expression may be monitored by a fluorescence detector. Also present in the cells is a control reporter system for assaying the activity of 5′ cap-dependent translation (the mechanism by which most cellular proteins are normally translated). The control reporter system contains a CMV promoter that drives the transcription of an RNA containing 5′ cap-dependent UTR, operably linked to an RNA encoding RFP (red fluorescent protein). Translation of RFP is dependent on the activity of the 5′ cap-dependent UTR.

Cells containing both the cyclic compound library and the reporter systems described above were first screened for survival upon exposure to diptheria toxin, and then screened by FACS to identify cells that had reduced GFP expression, as compared to RFP expression. Cells were expanded and replica-plated and grown in the presence or absence of dox (100 ng/ml) for four days. Plates were stimulated with IL-4 and GFP expression (relative to RFP) was monitored by flow cytometry. The IRES-inhibitory activity of a peptide is evaluated by calculating the ratio of the geometric mean of GFP expression of peptide positive cells (“peptide pos+” cells i.e., cells not contacted with dox) to the geometric mean of GFP expression of peptide negative cells (“peptide pos−” cells i.e., cells contacted with dox).

A ratio of 1.19 or below indicates that a particular cyclic peptide has no significant activity, whereas a score of at least 1.20 indicates that a particular cyclic peptide has significant IRES-inhibitory activity. Cyclic peptides assigned higher scores have a greater IRES-inhibitory activity.

Several IRES-inhibitory peptides were identified. The amino acid sequences of those IRES-inhibitory cyclic peptides were revealed by sequencing the nucleic acids encoding those peptides.

The screening assays identified cyclic peptides having the following amino acids sequences as having significant IRES-inhibitory activity: cyclo-CAW (1.37), cyclo-CMW (1.29), cyclo-CWW (1.41), cyclo-CYW (1.3), cyclo-SAW (1.21), cyclo-SFV (1.33), cyclo-SFW (1.55), cyclo-SIV (1.4), cyclo-SIW (1.36), cyclo-SKV (1.38), cyclo-SLW (1.27), cyclo-SMV (1.36), cyclo-SMW (1.28), cyclo-SVI (1.28), cyclo-SVV (1.64), cyclo-SVW (1.3), cyclo-SWF (1.33), cyclo-SWI (1.36), cyclo-SWM (1.22), cyclo-SWV (1.83), cyclo-SWW (2.37), cyclo-SWY (1.38), cyclo-SYV (1.4), cyclo-SYW (1.64), cyclo-TCW (1.48), cyclo-TFW (1.75), cyclo-THW (1.49), cyclo-TLW (1.2), cyclo-TMF (1.37), cyclo-TMV (1.56), cyclo-TMW (2.1), cyclo-TQW (1.7), cyclo-TSW (1.25) and cyclo-TYW (2.23). These cyclic peptides were produced by an intein having the junction sequence Val-His-Asn-X₁X₂X₃-Cys-Ile-Ser, where X₁ X₂ X₃ are the amino acids of the cyclic peptide.

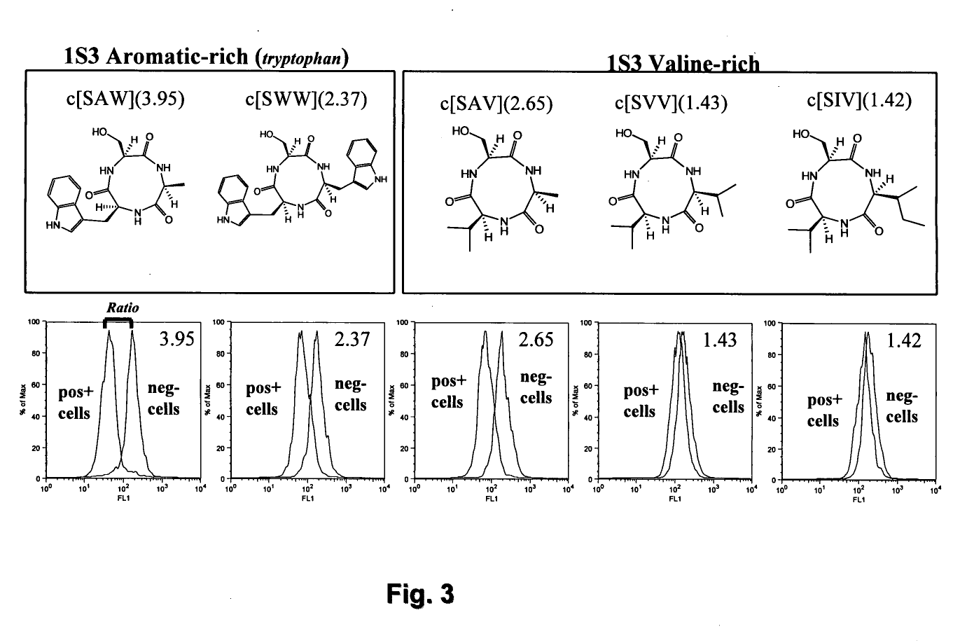

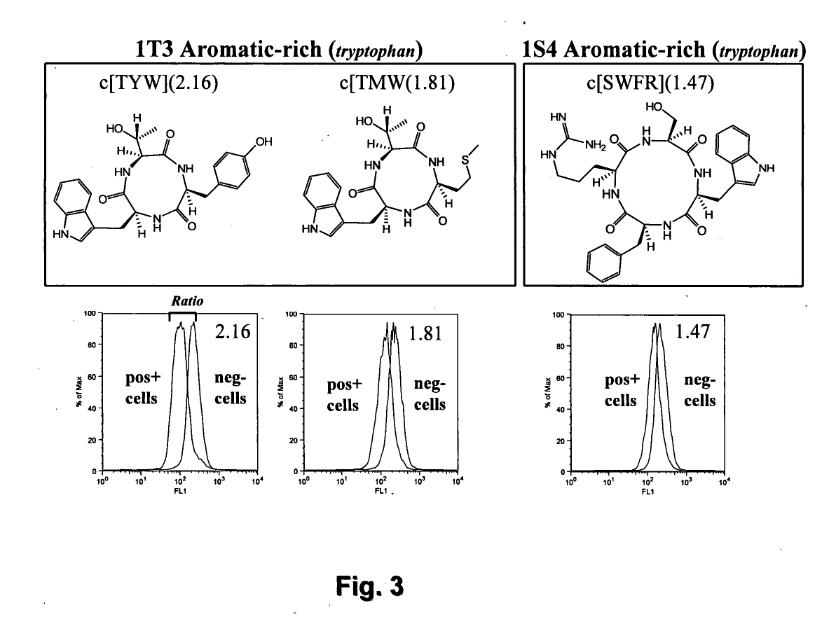

The amino acid sequence and structure of certain exemplary peptides, as well exemplary FACS results demonstrating an IRES inhibitory activity are illustrated in FIG. 3.

The following 3-mer peptides were also identified as having anti-IRES activity: cyclo-SAV (2.65), cyclo-SDV (1.23), cyclo-SEV (1.93), cyclo-SHV (1.91), cyclo-STV (2.24), cyclo-SAM (1.36), cyclo-SAT (1.21), cyclo-SAY (1.95) and cyclo-TAV (2.00). These 3-mer peptides were produced by an intein scaffold having a mutated sequence, as compared to the intein scaffold used to produce the library. For each of these peptides, the sequence of the junctions of the mutant scaffold is Val-His-Tyr-X₁X₂X₃-Cys-Ile-Ser, where X₁, X₂ and X₃ are the amino acids of the cyclic peptide. The significance of this amino acid change in the intein scaffold is not understood.

In addition, the following 3-mer peptides were also identified as having anti-IRES activity: cyclo-SAF (1.88), cyclo-SAI (1.30), cyclo-SAK (1.3), cyclo-SAQ (1.4), cyclo-SHW (2.26), cyclo-SPW (2.28), cyclo-SQW (2.20), cyclo-SRW (2.14), cyclo-STW (2.12), cyclo-SNW (2.10), cyclo-SSW (2.05), cyclo-SCW (2.01), cyclo-SEW (2.00), cyclo-SGW (1.94), cyclo-SDW (1.85), and cyclo-TAW (5.09). These 3-mer peptides were produced by an intein scaffold having a mutated sequence, as compared to the intein scaffold used to produce the library. For each of these peptides, the sequence of the junctions of the mutant scaffold is Val-His-Asn-X₁X₂X₃-Trp-Ile-Ser, where X₁, X₂ and X₃ are the amino acids of the cyclic peptide. The significance of this amino acid change in the intein scaffold is not understood.

The following 4-mer peptides were identified as having anti-IRES activity: cyclo-SWFR (1.44), cyclo-SWFA (1.31), cyclo-SWFK (1.58), cyclo-SWFM (1.31), cyclo-SWWR (2.03), cyclo-SWYR (1.49), cyclo-TWFR (1.56) and cyclo-SFWR (1.49). These 4-mer peptides were produced by an intein scaffold having a mutated sequence, as compared to the intein scaffold used to produce the library. For each of these peptides, the sequence of the junctions of the mutant scaffold is Val-His-Asn-X₁X₂X₃X₄-Cys-Ser, where X₁, X₂, X₃ and X₄ are the amino acids of the cyclic peptide. The significance of this amino acid change in the intein scaffold is not understood.

The number shown in parentheses after each of the amino acid sequences is the IRES-inhibitory activity ratio, as discussed above.

### Example 2

### Isolation of Peptides from Cells

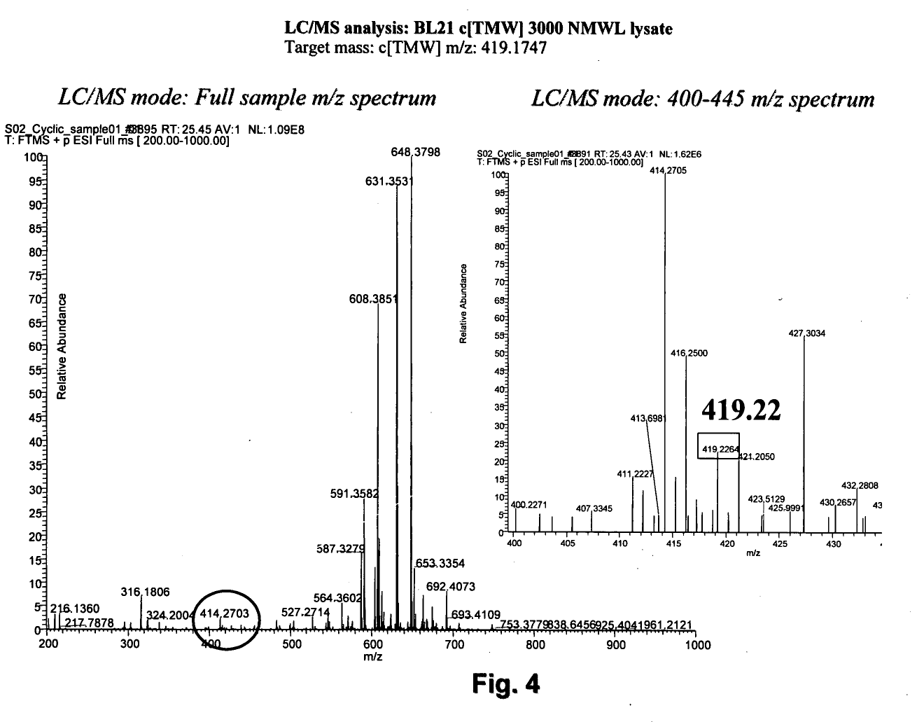

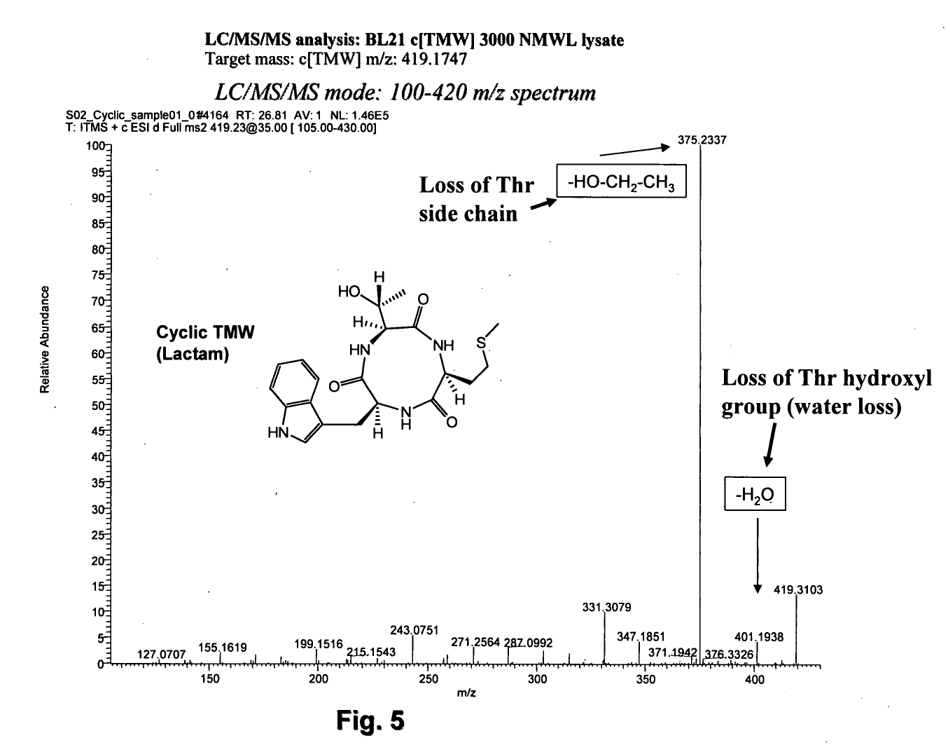

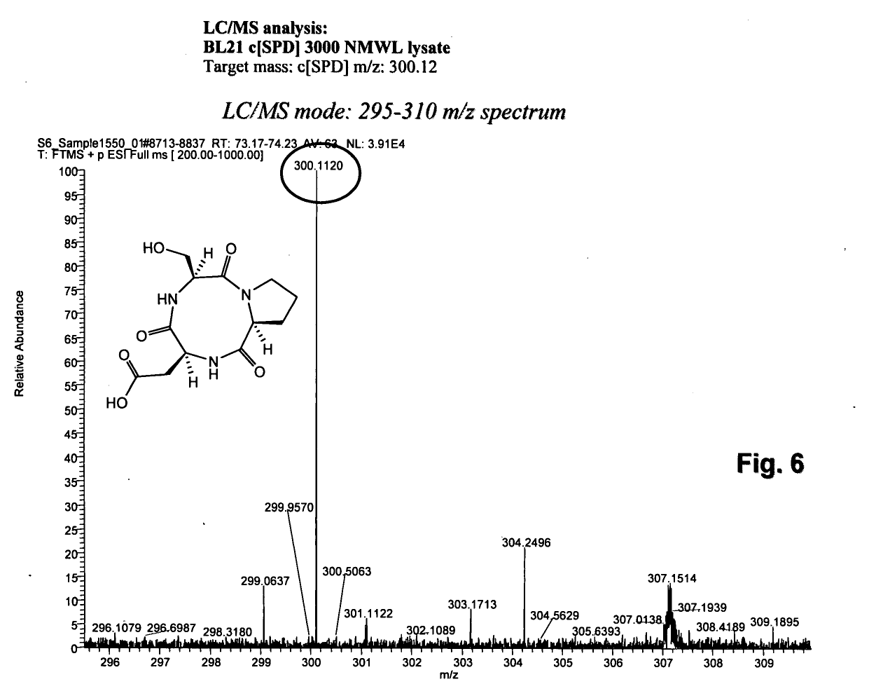

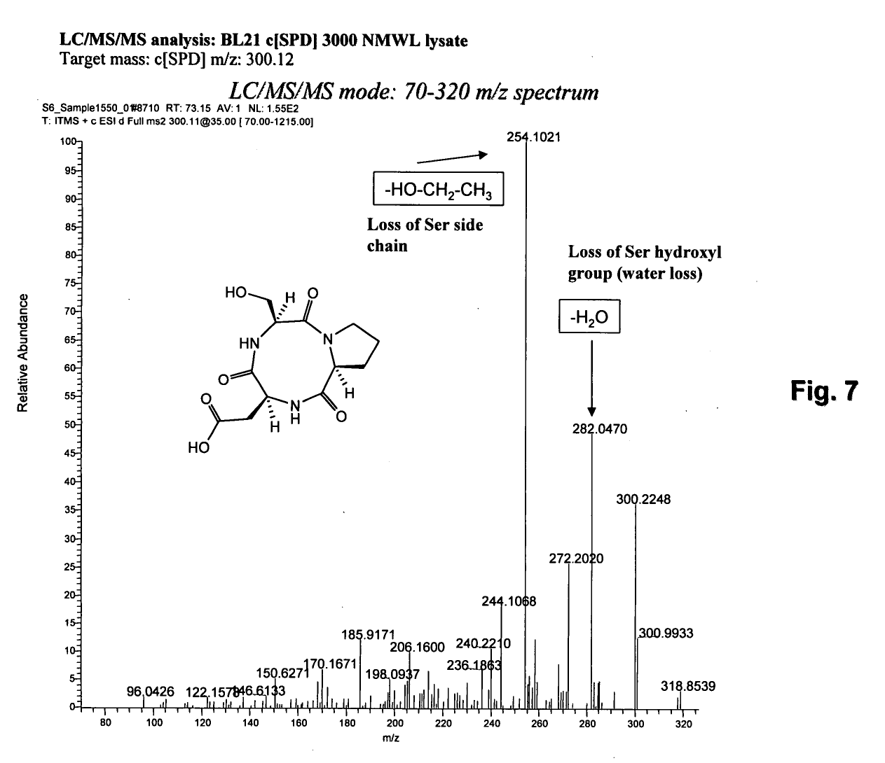

Library members that scored positive in the HCV IRES FACS assay were subcloned into the pGEX4T-1 expression vector as in-frame C-terminal fusions to Gluthione-S-transferase (GST) (Pfizer/Pharmacia, New York, N.Y.). DNA was transformed into BL21 bacteria (Stratagene, La Jolla, Calif.) and cells were cultured at 37° C. in 250 ml Terrific Broth with 100 μg Ampicillin until the optical density at 600 nm was 0.8. Expression was induced by addition of 0.5 mM isopropyl-beta-D-thiogalactopyranoside (IPTG) (Stratagene, La Jolla, Calif.) and cells were allowed to grow for an additional 12 hours at 16° C. Cells were collected by centrifugation and the cell pellet was overlaid with 10 ml ice cold Phosphate Buffered Saline (PBS) supplemented with 1× Complete Protease Inhibitor Cocktail (Roche Diagnostics GmbH, Mannheim, Germany). Cells were subjected to three freeze-thaw cycles at −80° C. and then sonicated for 2 minutes with a 550 Sonic Dismembrator set to power level 5 (Fisher Scientific, Hampton, N.H.). Cellular debris was removed by centrifugation and the clarified supernatant was subjected to filtration through a Microcon YM-3 regenerated cellulose 3000 Nominal Molecular Weight Limit (NMWL) centrifugation filter (Millipore, Bedford, Mass.). The filtered lysates were further separated by reverse phase chromatography on a C18 column into a MDS Sciex QStar pulsar QqTOF mass spectrometer. MS (FIGS. 4 and 6) and MS/MS spectrums (FIGS. 5 and 7) were examined for masses corresponding to the expected cyclic peptides.

FIGS. 4 and 5 show a compound (circled or boxed) having the predicted molecular weight of cyclo[TMW] (419.1747) in cell extracts. FIGS. 6 and 7 show a compound having the predicted molecular weight of cyclo[SPD] (300.12) in cell extracts.

### Example 3

### Isolation of Peptides Using N-Butanol

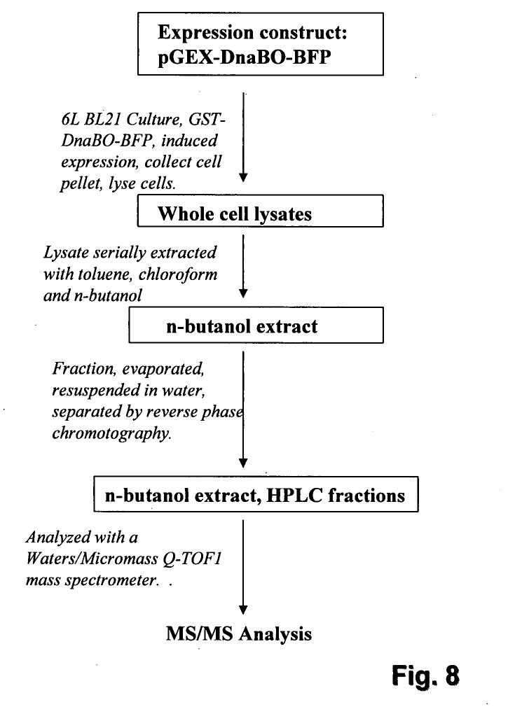

Library members that scored positive in the HCV IRES FACS assay were subcloned into the pGEX4T-1 expression vector as in-frame C-terminal fusions to Gluthione-S-transferase (GST) (Pfizer/Pharmacia, New York, N.Y.). DNA was transformed into BL21 bacteria (Stratagene, La Jolla, Calif.) and cells were cultured at 37° C. in 250 ml Terrific Broth with 100 g Ampicillin until the optical density at 600 nm was 0.8. Expression was induced by addition of 0.5 mM isopropyl-beta-D-thiogalactopyranoside (IPTG) (Stratagene, La Jolla, Calif.) and cells were allowed to grow for an additional 12 hours at 16° C. Cells were collected by centrifugation and the cell pellet was overlaid with 10 ml ice cold Phosphate Buffered Saline (PBS) supplemented with 1× Complete Protease Inhibitor Cocktail (Roche Diagnostics GmbH, Mannheim, Germany). Cells were subjected to three freeze-thaw cycles at −80° C. and then sonicated for 2 minutes with a 550 Sonic Dismembrator set to power level 5 (Fisher Scientific, Hampton, N.H.). Cellular debris was not removed and PBS was added to bring the final volume up to 250 ml. The whole cells lysates were mixed with an equal volume of toluene (250 ml), shaken vigorously for 2 minutes and then subjected to centrifugation to separate the phases. The aqueous phase was removed and then subjected to a second extraction with an additional 250 ml of fresh toluene. Both 250 ml toluene fractions were combined into a single vessel, evaporated under vacuum (about 10 mm Hg) until dry and the remaining material was resuspended in 2 ml of the starting solvent (toluene). The remaining aqueous phase was then subjected to additional phase extractions with both chloroform and n-butanol, respectively. The procedures for these extractions were identical to those used for toluene, with the exception that the dried n-butanol fraction was resuspended in 2 ml purified water instead of the starting extraction solvent of n-butanol. The peptides of interest were purified from other proteins and cell components using n-butanol. Samples were sonicated and then centrifuged to remove the insoluble precipitate. 20 μl of the extract was loaded onto a C18 reverse phase column and subjected to a 60 minute 2-60% gradient of acetonitrile in water and 0.1% trifluoroacetic acid (TFA). The mobile phase was collected in 1 ml fractions, lyophilized and resuspended in 100 μl of 66.9% water, 33% methanol, 0.1% acetic acid. Individual fractions were analyzed with a Waters/Micromass Q-TOF1 mass spectrometer. This method is illustrated in FIG. 8.

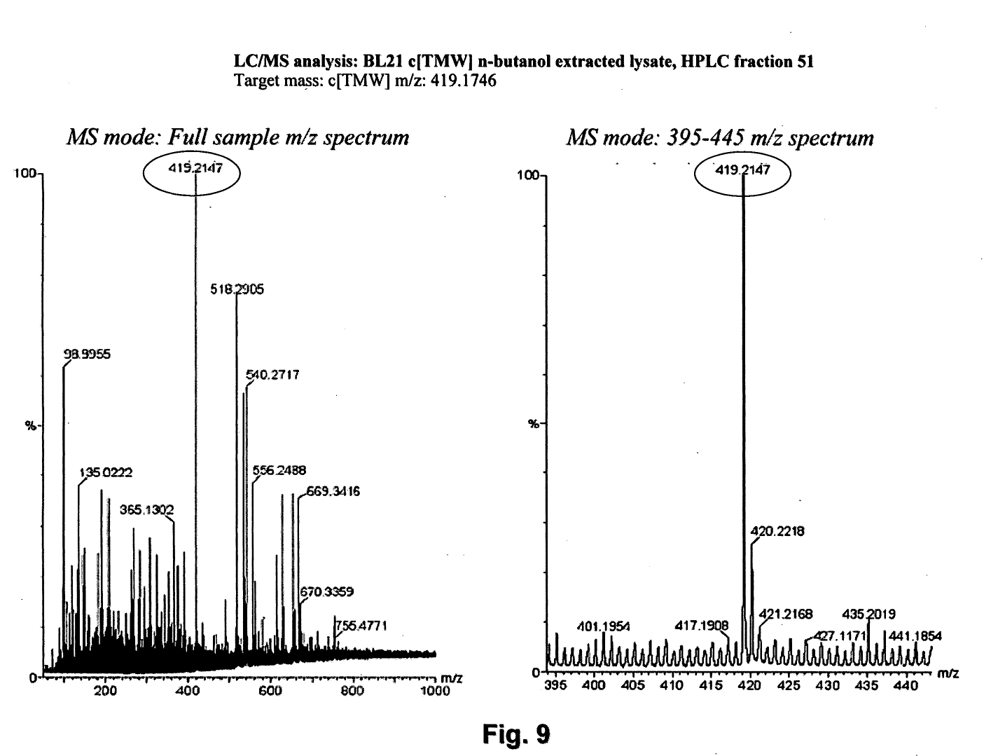

As shown in FIG. 9, serially extracted lysates from c[TMW]-expressing bacteria produced a single fraction, number 51, that contained a molecular ion corresponding to c[TMW] (C₂₀H₂₆N₄O₄S₁+H) at 419.2147. The observed mass of 419.2147 agreed well with the expected theoretical molecular ion mass of c[TMW] at 419.1747.

It is evident from the above discussion that the subject invention provides an important new means for inhibiting IRES-mediated translation. Since IRES-mediated translation is required for expression of a wide array of deadly virus, the above-described compounds may be used as effective anti-viral agents for those viruses. Further, since the above-described compounds are cyclic they are conformationally restricted and, as such, exhibit increased specificity and affinity in binding to other molecules, as compared to linear peptides. Further, the above-described cyclic peptides are thought to be more stable in cells and on the shelf than linear peptides, and may be small enough to avoid recognition by host immune system and to cross the plasma membrane of a cell. As such, the subject methods and compositions find use in a variety of different applications, including research, medical, therapeutic and other applications. Accordingly, the present invention represents a significant contribution to the art.

That which is claimed is:

1. A heterocyclic compound, wherein said compound is of the formula: 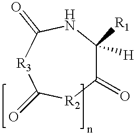
wherein n is 1 or 2, and
R₁ is hydroxymethyl, 1-hydroxyethyl or thiomethyl; and
R₂ and R₃ are each independently: 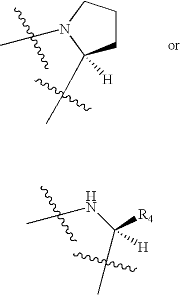
wherein R₄ is hydrogen, methyl, iso-propyl, iso-butyl, sec-butyl, methylthioethyl, benzyl, CH₂-linked 4-hydroxy-phenyl, CH₂-linked indole, hydroxymethyl, thiomethyl, ethanoic amide, propanoic amide, ethanoic acid, propanoic acid, 1-hydroxyethyl, 4-aminobutanyl, 4-(aminoiminomethyl)aminopropyl, hydroxymethyl, 1-hydroxyethyl, thiomethyl or CH₂-linked imidazole;
with the proviso that the compound is not a compound represented CAS registry numbers: 209353-30-0, 748142-25-8, 591781-32-7, 189179-32-6, 189179-28-0, 176703-10-9, 176703-09-6, 122886-11-7, 107208-67-3, 83797-39-1, 81017-86-9, 77782-99-1, 135432-38-1, 209353-31-1, or 189179-39-3.

2. The heterocyclic compound of claim 1,
wherein n is 2,
R₂ is: 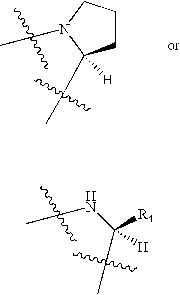
R₄ is hydrogen, methyl, iso-propyl, iso-butyl, sec-butyl, methylthioethyl, benzyl, CH₂-linked 4-hydroxy-phenyl, CH₂-linked indole, hydroxymethyl, thiomethyl, ethanoic amide, propanoic amide, ethanoic acid, propanoic acid, 1-hydroxyethyl, 4-aminobutanyl, 4-(aminoiminomethyl)aminopropyl, hydroxymethyl, 1-hydroxyethyl, thiomethyl or CH₂-linked imidazole and
R₃ is: 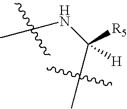
and R₅ is iso-propyl, sec-butyl, methylthioethyl, benzyl, CH₂-linked 4-hydroxy-phenyl, CH₂-linked indole, propanoic amide or 4-aminobutanyl.

3. The heterocyclic compound of claim 2,
wherein n is 2,
R₂ is: 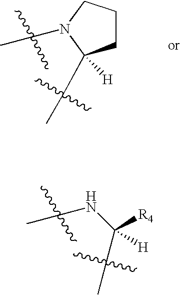
R₄ is hydrogen, methyl, iso-propyl, iso-butyl, sec-butyl, methylthioethyl, benzyl, CH₂-linked 4-hydroxy-phenyl, CH₂-linked indole, hydroxymethyl, thiomethyl, ethanoic amide, propanoic amide, 1-hydroxyethyl, 4-aminobutanyl, 4-(aminoiminomethyl)aminopropyl, hydroxymethyl, 1-hydroxyethyl, thiomethyl or CH₂-linked imidazole and
R₃ is: 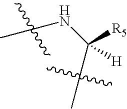
and R₅ is iso-propyl or CH₂-linked indole.

4. A heterocyclic compound that exhibits IRES-inhibitory activity, wherein said compound is of the formula:
cyclo[X₁X₂X₃X₄],
wherein X₄ may be present or absent, wherein X₁ is a Ser, Thr or Cys amino acid,
wherein X₂, X₃, and X₄ are independently any naturally-occurring amino acids or a non-natural amino acid selected from the group consisting of: β-alanine (β-Ala), 2,3-diaminopropionic acid (Dpr), 4-aminobutyric acid, α-aminoisobutyric acid (Aib); ε-aminohexanoic acid (Aha); δ-aminovaleric acid (Ava); N-methylglycine, sarcosine (MeGly), ornithine (Orn), citrulline (Cit), t-butylalanine (t-BuA), t-butylglycine (t-BuG), N-methylisoleucine (MeIle), phenylglycine (Phg), cyclohexylalanine (Cha), norleucine (Nle), 2-naphthylalanine (2-Nal), 4-chlorophenylalanine (Phe(4-Cl)), 2-fluorophenylalanine (Phe(2-F)), 3-fluorophenylalanine (Phe(3-F)), 4-fluorophenylalanine (Phe(4-F)), penicillamine (Pen), 1,2,3,4-tetrahydroisoquinoline-3-carboxylic acid (Tic), β-2-thienylalanine (Thi), methionine sulfoxide (MSO), homoarginine (hArg), N-acetyl lysine (AcLys), 2,3-diaminobutyric acid (Dab), 2,4-diaminobutyric acid (Dbu), p-aminophenylalanine (Phe(pNH₂)), N-methyl valine (MeVal), homocysteine (hCys), p-methyl cysteine and homoserine (hSer), and
wherein the amino acids of the compound are joined by peptide bonds,
with the proviso that the compound is not a compound represented CAS registry numbers: 209353-30-0, 748142-25-8, 591781-32-7, 189179-32-6, 189179-28-0, 176703-10-9, 176703-09-6, 122886-11-7, 107208-67-3, 83797-39-1, 81017-86-9, 77782-99-1, 135432-38-1 209353-31-1, or 189179-39-3.

5. A composition for treating a viral infection, comprising:
a therapeutically effective amount of a heterocyclic compound of claim 1; and
a pharmaceutically acceptable excipient.

6. A kit for treating a viral infection, comprising:
the composition of claim 5; and
instructions for treating said viral infection using said composition.

7. A method for inhibiting viral IRES-mediated translation, comprising:
contacting a viral IRES with a heterocyclic compound of claim 1 to inhibit translation from said IRES.

8. The method of claim 7, wherein said viral IRES is present in a cell-free environment.

9. The method of claim 7, wherein said viral IRES is present in a cell and said method involves contacting said cell with said heterocyclic compound.

10. The method of claim 9, wherein said cell is a mammalian cell.

11. A method for inhibiting cellular IRES-mediated translation, comprising:
contacting said IRES with a heterocyclic compound of claim 1 to inhibit translation from said IRES.

12. A method of inhibiting viral replication in a virus-infected cell, comprising:
contacting said cell with a heterocyclic compound of claim 1 to inhibit viral replication in said cell.

13. The method of claim 12, wherein said virus is a Flaviviridae virus.

14. The method of claim 12, wherein said virus is hepatitis C virus (HCV).

15. The method of claim 12, wherein said cell is a cell in vitro.

16. The method of claim 12, wherein said cell is a cell in vivo.

17. The method of claim 12, wherein said cell is in an organ removed from a mammalian subject.

18. The method of claim 17, wherein said organ is a liver.

19. A method of treating a subject for a viral infection, comprising:
administering to said subject a formulation comprising a heterocyclic compound of claim 1 in an amount effective to treat said subject for said viral infection.

20. The method of claim 19, wherein said subject is infected with a Flaviviridae virus.

21. The method of claim 19, wherein said subject is infected with HCV.
